(19) United States
(12) Patent Application Publication
(10) Pub. No.: US 2023/0225892 A1
(43) Pub. Date: Jul. 20, 2023

(54) SYSTEMS AND METHODS FOR IMPLANTING A GASTRIC BYPASS DEVICE

(71) Applicant: Quipmen Medical Inc., Thousand Oaks, CA (US)

(72) Inventors: KAPIL GUPTA, Santa Monica, CA (US); RAVI SAWHNEY, THOUSAND OAKS, CA (US); ALEX GILBERT, McLean, VA (US); JOHN VERNON, Malibu, CA (US); CARY CHOW, Santa Monica, CA (US); JOHN ELAM, Woodland Hills, CA (US)

(73) Assignee: Quipmen Medical Inc., Thousand Oaks, CA (US)

(21) Appl. No.: 18/125,249

(22) Filed: Mar. 23, 2023

Related U.S. Application Data
(63) Continuation-in-part of application No. 17/207,718, filed on Mar. 21, 2021.
(60) Provisional application No. 62/992,311, filed on Mar. 20, 2020.
(60) Provisional application No. 63/073,154, filed on Sep. 1, 2020.

(51) Int. Cl. A61F 5/00; A61B 17/11

(52) U.S. Cl. CPC A61F 5/0089; A61B 17/1114; A61B 2017/00535

(57) ABSTRACT

The present invention relates to a system for bypassing stomach and creating a barrier for the proximal small bowel to include duodenum and jejunum so that food delivered is directly in the jejunum that results in weight loss. The technique involves placement and fixation of a gastric bypass device within the stomach to cover the small bowel. The present invention also provides a device and method which enables the suturing of the gastric bypass device. The duodeno-jejunal barrier remains unchanged and helps in bypassing the absorptive surface of the proximal small bowel.

### CROSS-REFERENCE TO RELATED APPLICATIONS

This is Continuation-in-Part application of U.S. Ser. No. 17/207,718, filed on Mar. 21, 2021, which is a non-provisional application of U.S. Provisional Application No. 62/992,311, filed on Mar. 20, 2020, and of U.S. Provisional Application No. 63/073,154, filed on Sep. 1, 2020, the entirety of which are incorporated herein by referenced.

### FIELD OF INVENTION

The present invention is generally related to implantable weight control devices and, more specifically, to a device and a method that restricts the stomach and creates a barrier for a proximal small bowel to include duodenum and jejunum so that the food is delivered in the jejunum, ultimately resulting in weight loss.

### BACKGROUND OF THE INVENTION

For weight loss and control of metabolic diseases like diabetes mellitus, various invasive and non-invasive techniques have been performed and tried. These can range from all the way from surgical interventions like Roux-en-Y Gastric Bypass, Vertical sleeve Gastrectomy and Lap Band placement etc. Lifestyle modification and pharmacotherapy continues to be an option for individuals averse to surgical management. There are newer endoscopic techniques like vertical sleeve gastroplasty, intragastric balloons, and small bowel sheath etc.

In general, lifestyle modification primarily dietary management with exercise regimen and pharmacotherapy is usually the first modality recommended for patients in the BMI range of 30 to 40. These interventions are limited by regaining weight as soon as the aggressive measures are stopped. Various surgical procedures available in the medical industry include laparoscopic and open Roux-en-Y gastric bypass. Some of the less invasive surgical interventions include sleeve gastrectomy, which can be performed laparoscopically and adjustable gastric band, duodenal switch, and duodenal pancreatic diversion etc.

All the surgical maneuvers do provide a sustained and effective weight loss but the adoption has been limited to only providing surgical management for less than 1% of patients who meet the criteria for surgical management. Even after significant data, the benefits of surgical management and the adoption has been unsatisfactory. Some of the reasons for poor adoption are reluctance of having surgery, early and delayed complications. With advancement and refinement of surgery, even though the mortality rates are significantly lower but the morbidity continues to be significant.

All the above factors lead to a significant proportion of the population which continues to be in the overweight and obese category. This leads to significant other co-morbidities like diabetes mellitus, fatty liver disease, sleep apnea, osteoarthritis, and other significant cardiovascular diseases, which can be preventable. These metabolic diseases, if managed appropriately with weight reduction can lead to significant cost savings for healthcare.

To make a significant impact, an obesity more non-surgical options needed. It is very likely that if non-invasive, non-surgical options found to be more or equally effective, the adoption will be much higher. Some of the non-surgical options that have gained validation are gastric balloons, and recently popularized endoscopic sleeve gastroplasty. All these mentioned non-surgical intervention and tools only affect one mode of weight loss.

A U.S. issued U.S. Pat. No. 9,795,498, assigned to Apollo Endosurgery Inc., describes an transorally implanted intragastric balloon for weight control in which a variable size balloon with one or interconnected regions acting to exert a pressure on the stomach to provide a stomach volume occupying effect and to anchor the balloon within the stomach. Although the transoral intragastric device placed in the patient's stomach through the mouth and the esophagus and then being placed to reside in the stomach. However, the intragastric balloons are space-occupying objects in the stomach that lead to early satiety. Nevertheless, the intragastric balloons have been poorly tolerated because of significant post-procedure symptoms like nausea and vomiting. Many times this requires early removal of the balloon.

Another prior art referenced, U.S. Pat. No. 7,220,284, assigned to ValenTx Inc., discloses apparatus and method for treatment of morbid obesity using minimally invasive techniques. The apparatus includes a system of components including an artificial stoma device, a gastric sleeve device, an intestinal sleeve device and a combined gastrointestinal sleeve device. Although the apparatus described here effectively reduces stomach volume, bypassing a portion of the stomach and small intestines, reducing nutrient absorption in the stomach and small intestines, and depositing minimally or undigested food farther than normal into the intestines, thereby stimulating intestinal responses. However, the technique of delivering the gastrointestinal sleeve device described is inefficient and incompetent.

Another publication, U.S. Patent Publication No. 2020/0179149 assigned to the ValenTx Inc., disclosed devices and methods for attachment of an endolumenal gastrointestinal device such as an artificial stoma device, a gastrointestinal bypass sleeve or other therapeutic or diagnostic device within a patient's digestive tract. In one of the applications of the invention, an endolumenal bypass sleeve is removable attached in the vicinity of the gastroesophageal junction to treat obesity and its co-morbidities including diabetes. However, the endolumenal bypass sleeve that described is a duodenojejunal barrier is reported in some trials, the endolumenal bypass sleeve described and method of delivering the same is inefficient and incompetent.

Therefore, there is a need for a minimal invasive endoscopic technique which is somewhat reversible and combines three methods of weight loss including restrictive by decreasing capacity of the stomach, decreasing the motility of the stomach and bypassing the small bowel. Another need is to provide a primary method to bypass the proximal small bowel but it shall not make any changes to the stomach.

Hence, there is a need for a simpler, minimally invasive, near reversible device and method to assist in weight loss which combines different mechanisms like restrictive binding, decreasing motility and mal-ab sorption.

It is apparent now that numerous methods and systems are developed in the prior art that are adequate for various purposes for which they were devised. Furthermore, even though these inventions may be suitable for the specific purposes to which they address, accordingly, they would not be suitable for the purposes of the present invention as heretofore described. Thus, there is a need for a minimal invasive technique for implanting a gastric bypass device that provides weight loss by restrictive mechanism by decreasing the size of the stomach and decreasing absorption by creating a duodenojejunal barrier.

### SUMMARY OF THE INVENTION

The present invention provides a system for implanting a gastric bypass device at a desired position within stomach of a patient. The system includes a catheter assembly with a proximal end and a distal end. The system further includes a capsule body with a first end and a second end. The first end of the capsule body connected to the distal end of the catheter assembly.

The capsule body further includes a hollow lumen configured for holding a folded sleeve with the gastric bypass device. The capsule body further includes a spring wire support placed within internal periphery of the capsule body to hold the gastric bypass device within the folded sleeve, which is further expanded to release the gastric bypass device from the folded sleeve further implanted within the stomach of the patient. The capsule body further includes a capsule cover with a one end and another end. The capsule cover is connected to the second end of the capsule body through the one end.

The system further includes a flexible catheter connected to another end of the capsulebody. The flexible catheter further includes a first hole at other side of the flexible catheter to receive a snare wire. The snare wire is received by the first end of the capsule body via the capsule cover through the first hole of the flexible catheter. The flexible catheter further includes a pair of second holes at the other side of the flexible catheter to receive a push wire from each of the pair of second holes. Further, each of the push wires is received at the first end of the capsule body via capsule cover.

The flexible catheter further includes a third hole, at the other side to receive a guide wire. The guide wire is received at the center of the proximal end of the catheter assembly via the capsule body and the capsule cover. The flexible catheter further includes a central lumen extending between the capsule cover and the other end of the flexible catheter for transferring a fluid to the folded sleeve within the capsule body via the capsule cover.

The system further includes a motorized controller, which is coupled to the flexible catheter. The motorized controller is configured for controlling precise advancement and retraction of the guide wire, the push wire, the snare wire and transfer of the fluid into the folded sleeve. Further, the advancement and retraction of the of the guide wire, the push wire, the snare wire disengages the capsule body from the catheter assembly to unfold the folded sleeve. Further, the transfer of fluid inflates the unfolded sleeve. The fluid is transferred from the controller via the flexible catheter to the capsule body for inflating the unfolded sleeve. Further, the push wires and the snare wires are advanced and retracted, followed by disengagement of the capsule body from the inflated sleeve.

The system further includes a base station having a plurality of independently controllable and coaxially aligned drive drafts, each of the draft shafts having a reel affixed thereto, the drive shafts being operable for independently controlling at least one of the guide wire, snare wire, and push wires. The controller also including a bladder and peristaltic pump, the peristaltic pump being controllable for forcing fluid from the bladder into the folded sleeve.

Furthermore, the guide wire and the push wires are held in place and the snare wire is withdrawn to disengage the catheter assembly, thereby releasing the inflated sleeve from one end. Further, the gastric bypass device is released upon full disengagement of the capsule body from the inflated sleeve. Further, the gastric bypass device is implanted at the desired position in the stomach. The controller may be automatic or manual. In one aspect of the present invention, the controller is an electrically controlled electrical device.

A primary objective of the present invention is to provide a minimally invasive modality of a gastric bypass device to enhance the weight loss.

An objective of the present invention is to provide a gastric bypass device that provides weight loss by restrictive mechanism by decreasing the size of the stomach.

Another objective of the present invention is to provide a gastric bypass device that provides weight loss by decreasing absorption by creating a duodenojejunal barrier.

Yet another objective of the present invention is to provide a gastric bypass device that does not require no external incision thus minimal infectious complications.

Lastly, the objective of the present invention is to provide a minimally invasive device, which is adapted to provide nutrition directly in the jejunum, bypassing the absorptive surface of the proximal small bowel.

In one embodiment of the present invention, the catheter assembly includes a nose cone insert with a front portion and a back portion. The back portion of the catheter assembly further connected to the capsule body. The back portion of the nose cone insert retains the folded sleeve in position by the engagement of the snare wire and the push wires. The snare wire is bifurcated, and then further inserted, which pass through a small hole in each pair of the push wires diametrically opposed, forming a triangular structure for retaining the folded sleeve at the first end of the capsule body.

The nose cone insert further allows the guide wire to pass through the hole in the second side opposite the first side of the nose cone insert. The catheter assembly further includes a nose cone for enclosing the nose cone insert. The nose cone further includes an opening which overlaps with the hole of the nose cone insert for holding the guide wire.

In another embodiment of the present invention, the nose assembly in this embodiment is preferably a biodegradable or resorbable material such as polycaprolactone, polyglecaprone, polyglactin, or other known biodegradable or resorbable material. More preferably, the biodegradable or resorbable material is polycaprolactone. The selection of the biodegradable or resorbable material coordinated with the natural pH of the anatomical environment of the stomach, to achieve a predetermined sequential degradation of the catheter assembly after releasing from the expanded or inflated sleeve, thereby allowing proper implantation of the gastric bypass device within the stomach of the patient.

In one another embodiment of the present invention, the capsule body coupled to the proximal end of the catheter assembly. The capsule body includes a hollow lumen configured for holding a folded sleeve with the gastric bypass device. In one aspect of the present invention, the sleeve folded several times transversely. Further, the sleeve placed longitudinally within the hollow lumen of the capsule body. More preferably, the folded sleeve includes a proximal end and distal end. The proximal end of the folded sleeve includes the gastric bypass device held at place by the spring wire support. The distal end of the folded sleeve is fixed to the back portion of the nose cone insert of the catheter assembly.

In another aspect of the present invention, the capsule body further includes a spring wire support placed within internal periphery of the capsule body to hold the gastric bypass device with the proximal end of the folded sleeve. The spring wire support is preferably made of a nitinol material or similar material.

In one another aspect of the present invention, the capsule body further includes multiple channels longitudinally arranged around the folded sleeve to place each of the snare wire, the push wire and the guide wire coming from the flexible catheter to the first end of the capsule body. The snare wire and the push wires converge at the first end of the capsule body and the back portion of the nose cone insert of the catheter assembly. Further, the guide wire leads to the hole of the catheter assembly.

In yet another aspect of the present invention, the capsule body, the folded sleeve and the gastric bypass device is made of polytetrafluoroethylene, polyethylene, cast PTFE, Teflon, fluorinated ethylene propylene, perfluoroalkoxy, PTFE, PFA, extruded FEP and extruded PFA, extruded PTFE, silicone. More preferably, the folded sleeve and the gastric bypass device of the present application are made up of polyethylene.

In yet another embodiment of the present invention, the flexible catheter connected to another end of the capsule cover. The flexible catheter is made of silicone or similar material.

In one embodiment of the present invention, the controller is hand-held controller. More specifically, the controller shaped like a handgun. In one aspect of the present invention, the controller further includes a gun-shaped member with a proximal end and a distal end. More preferably, the gun-shaped member receives the flexible catheter from the proximal end towards the distal end of the gun-shaped member.

The controller further includes a spooler with multiple channels arranged to hold each of the guide wire and the snare wire separately between the pair of push wires. The spooler further attached to the distal end of the gun-shaped member of the spooler. The spooler further includes a multiple wire guiding tubes for receiving each of the guide wire, the snare wire and the pair of push wires separately from the respective channels of the spooler. More specifically, there are four wire-guiding tubes each for the snare wire, guide wire and the pair of the push wires. Each of the guide wire, the snare wire and the pair of push wires separately pass from the spooler via the multiple channels within the wire guiding tube and towards the flexible catheter.

The spooler further includes a multiple contra-rotating friction roller is placed at bottom of each channels for feeding the wires to the respective wire guiding tubes. The contra-rotating friction roller pressed against the spooler to advance or retract one of the guide wire, the snare wire and a pair of push wires into the wire guiding tubes respectively. Further, the contra-rotating friction roller released against the spooler one of the guide wire, the snare wire and a pair of push wires into the wire guiding tubes respectively.

In one another embodiment of the present invention, the controller further includes a fluid transfer tube placed between the guide wire and the snare wire within a channel out of multiple channels. The fluid transfer tube further connected to a fluid inlet, which is at a rear end of the spooler. The fluid transfer from the fluid transfer tube controlled from a peristaltic pump rotor placed on the channel below the fluid transfer tube. The multiple rollers placed at periphery of the peristaltic pump rotor to regulate the transfer of fluid from the fluid transfer tube.

In one another embodiment of the present invention, the controller further includes a multiple function selectors linearly arranged at the proximal end of the gun-shaped member. Out of multiple function selectors, multiple mode selector provided to select operation of the guide wire, the snare wire, the pair of push wires and the fluid transfer tube. Out of multiple function selectors, a mode selector provided to select precise advancement or retraction of one of the guide wire, the snare wire, and the pair of push wires. In one aspect of the present invention, the function selector may be buttons or turn knobs.

In yet another embodiment of the present invention, the controller further includes a trigger for initiating action based on selection from the function selectors. The action is either advancement or retraction of one of the guide wire, the snare wire or the pair of push wires or the transfer of fluid from the fluid transfer tube. In one aspect of the preset invention, the trigger is either an actuator lever or a hand crank knob.

The advancement and retraction of the push wire and the snare wire lead the way for disengaging the capsule body from the catheter assembly, followed by the unfolding of the folded sleeve. Once the folded sleeve is fully unfolded, the fluid transferred from the controller to the capsule body via the central lumen of the flexible catheter and the capsule cover. The transfer of fluid leads to the expansion or inflation of the unfolded sleeve.

Once the folded sleeve fully expands or inflates, the inflated sleeve fully disengages from the capsule body to be released from the expanded folded sleeve to release the gastric bypass device along with the spring wire support. Further, the guide wire removed followed by the snare wire, and then the push wires removed from the stomach of the patient. Further, the distal end of the inflated sleeve is released from the proximal end of the catheter assembly, thereby releasing and further deployment of the gastric bypass device within the stomach of the patient.

In one embodiment of the present invention, the present disclosure provides a method of implanting a gastric bypass device within a stomach of a patient. The method includes advancement of a guide wire through an esophagus into the stomach and upper digestive tract of the patient from a catheter assembly by a controller. The method further includes placement of a capsule body that connected with the catheter assembly at one end near the desired position. The method further includes advancement of a pair of push wires and a snare wire in the capsule body by the controller. The method further includes withdrawal of the capsule body from the catheter assembly to unfold a sleeve with a gastric bypass device within the capsule body.

The method further includes injection of a fluid from a fluid transfer tube of the controller via a lumen of a flexible catheter, further attached to a capsule cover at other end of the capsule body. The method further includes inflation of the sleeve within the capsule body with the fluid to form an inflated sleeve. The method further includes disengagement of the inflated sleeve from the capsule body.

The method further includes removal of the guide wire followed by the snare wire and the pair of push wires by the controller to release the catheter assembly from the inflated sleeve. The method further includes releasing a spring wire support holding the gastric bypass device from the inflated sleeve. The method further includes emerging of the gastric bypass device to acquire a funnel shape. The method further includes suturing of the gastric bypass device within the stomach of the patient at the desired position.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

Although, the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

The presence of broadening words and phrases such as “one or more,” “at least,” “but not limited to” or other like phrases in some instances shall not be read to mean that the narrower case is intended or required in instances where such broadening phrases may be absent.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 illustrates a system for implanting a gastric bypass device in accordance with the present invention;

FIG. 2 illustrates a front part of the system for implanting the gastric bypass device in accordance with the present invention;

FIG. 3A illustrates a nose cone insert of a catheter assembly in the front part in accordance with the present invention;

FIG. 3B illustrates a nose cone enclosing the nose cone insert in accordance with the present invention;

FIG. 4A illustrates a cross-sectional view of the front part of the system for implanting the gastric bypass device in accordance with the present invention.

FIG. 4B illustrates a cross-sectional view of the front part of the system for implanting the gastric bypass device showing a partially unfolded sleeve in accordance with the present invention;

FIG. 4C illustrates an inflated sleeve of the capsule body connected to the catheter assembly from one end in accordance with the present invention;

FIG. 4D illustrates a cross-sectional view of the inflated sleeve outside the capsule body and attached to a gastric bypass device within the capsule body in accordance with the present invention;

FIG. 4E illustrates a cross-sectional view of the gastric bypass device being released from the capsule body in accordance with the present invention;

FIG. 4F illustrates a view of the release of the catheter assembly from the inflated sleeve in accordance with the present invention;

FIG. 4G illustrates a view of the funnel shaped gastric bypass device with the inflated sleeve in accordance with the present invention;

FIG. 4H illustrates a cross-sectional view of the funnel shaped gastric bypass device with the inflated sleeve in accordance with the present invention;

FIG. 5 illustrates a view of the front part of the system showing the capsule cover in detail in accordance with the present invention;

FIG. 6A illustrates a view of a flexible catheter connected to the capsule cover depicting a central lumen and a multiple holes in accordance with the present invention;

FIG. 6B illustrates a cross-sectional view of a flexible catheter connected to the capsule cover depicting a central lumen and multiple holes in accordance with the present invention;

FIG. 7A illustrates a view of a hand-held controller in accordance with the present invention;

FIG. 7B illustrates a cross-sectional view of the hand-held controller showing a spooler in accordance with the present invention;

FIG. 7C illustrates a perspective view of the spooler in accordance with the present invention;

FIG. 7D illustrates a top cross-sectional view of the hand-held controller in accordance with the present invention;

FIG. 7E illustrates a cross-sectional back view of the spooler in accordance with the present invention;

FIG. 7F illustrates a front view of the spooler in accordance with the present invention;

FIG. 7G illustrates a mid-lane cross-sectional view of the hand-held controller in a fluid-transfer mode in accordance with the present invention;

FIG. 7H illustrates a horizontal cross-sectional view of a mode selection buttons through axis of mode selection buttons in accordance with the present invention;

FIG. 8 illustrates a method for implanting a gastric bypass device at a desired position within a stomach of a patient in accordance with the present invention;

FIG. 9 illustrates an automated version of the controller in accordance with various embodiments of the present invention;

FIG. 10 illustrates a base station as used in the automated version of the controller;

FIG. 11 is an interior-view of the base station;

FIG. 12 is an interior-view of the base station in accordance with various embodiments of the present invention;

FIG. 13 is an interior-view of the base station in accordance with various embodiments of the present invention, depicting drive shafts;

FIG. 14 is an interior-view of a reusable base component in accordance with various embodiments of the present invention;

FIG. 15 is an exploded-view illustration of the drive shafts;

FIG. 16 is an exploded-view illustration of the wire reels; and

FIG. 17 is an illustration depicting progressive assembly of the wire reels onto the drive shafts.

### DETAILED DESCRIPTION

The present invention is generally related to implantable weight control devices and, more specifically, to a device and a method that creates a barrier for a proximal small bowel to include duodenum and jejunum so that the food is delivered in the jejunum, ultimately resulting in weight loss. The following description is presented to enable one of ordinary skill in the art to make and use the invention and to incorporate it in the context of particular applications. Various modifications, as well as a variety of uses in different applications, will be readily apparent to those skilled in the art, and the general principles defined herein may be applied to a wide range of aspects. Thus, the present invention is not intended to be limited to the aspects presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112(f). In particular, the use of “step of” or “act of” in the claims herein is not intended to invoke the provisions of 35 U.S.C.

As noted above, the present invention provides a system for implanting a gastric bypass device at a desired position within stomach of a patient. The normal anatomy of the upper gastrointestinal tract is the primary area that is modified or bypassed. The body of the stomach, which has two sides, the lesser curvature and the greater curvature, follows the fundus. The segment after the body of the stomach is the antrum, which connects with the small bowel. The opening between the antrum and the small bowel is the pylorus, which changes into the duodenal bulb. The second portion of the duodenum, the third portion of the duodenum and the fourth portion of the duodenum form the first few feet of the small bowel. Significant absorption of nutrients happens in these different parts of the duodenum. The duodenum loops into the jejunum by making a sharp angulated turn, which is pulled up by the ligament of treitz. This then changes into jejunum where primary absorption happens. Thus, although not limited thereto, the desired implant site is approximately 5-10 centimeters from the gastro-esophageal junction and spares the fundus. Further details are provided below in conjunction with the figures submitted herewith.

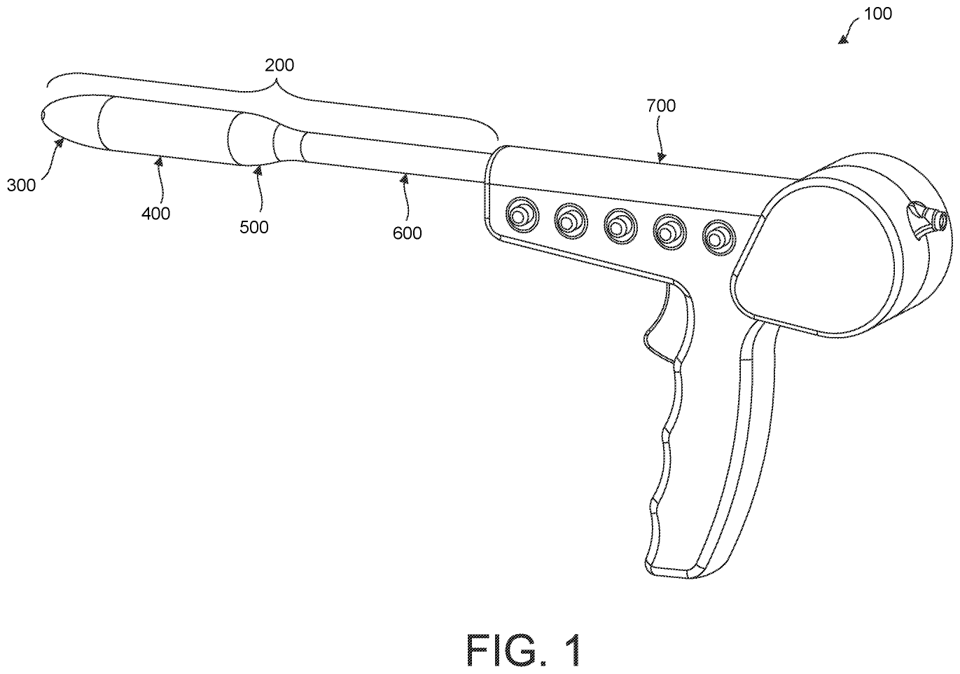

FIG. 1 represents an illustrative view of a system 100 configured for deploying and implanting a gastric bypass device within the stomach of the patient, depicting the system 100 prior to deployment. The system (100) includes a first part (200) and a controller (700). The first part (200) of the system (100) includes a catheter assembly (300) having a proximal end and a distal end, which is configured for holding a guide wire, which is further advanced and retracted to move the entire system in a particular direction as the gastric bypass device is deployed at desired specific position.

The first part (200) of the system (100) further includes a capsule body (400) having a first end and a second end. The first end of the capsule body (400) is connected to the proximal end of the catheter assembly (300). The system (100) further includes a capsule cover (500) having a one end and another end. The first part (200) of the system (100) further includes a capsule cover (500) connected to the second end of the capsule body (400) through the one end. The first part (200) of the system (100) further includes a flexible catheter (600) connected to another end of the capsule cover (500).

The system (100) further includes a controller (700) that is detachably coupled to the flexible catheter (600) of the first part (200) of the system (100). The controller (700) provides controlling commands for controlling the advancement and retraction of the guide wire, the push wire, and the snare wire for disengagement of the capsule body (400) from the catheter assembly (300).

Further, the controller (700) provides components for transferring the fluid into the sleeve, thereby expanding and disengaging the sleeve from the capsule body (400) and the catheter assembly (300). Through expansion of the sleeve, the gastric bypass device is allowed to be released from the sleeve and deployed at the desired position in the stomach. Thus, the system (100) is used for deploying the gastric bypass device through the esophagus into the stomach and upper digestive tract along a flexible guide wire that is integrated in the system (100), which is advanced and retracted by means of the controller (700).

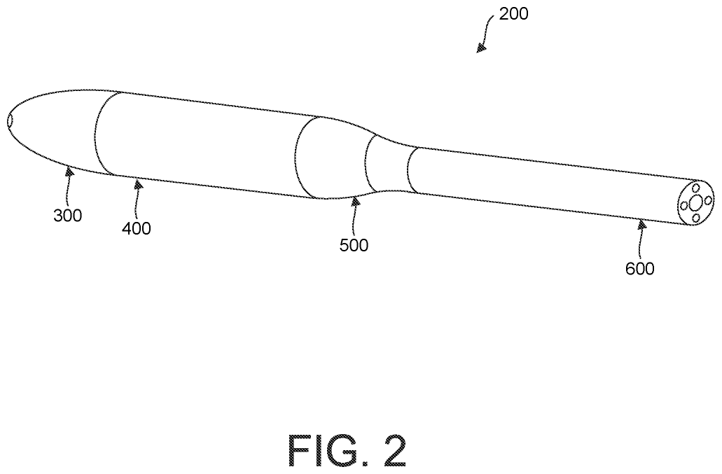

FIG. 2 represents an illustrative view of the first part (200) of the system (100), which is configured for deploying and implanting a gastric bypass device within the stomach of the patient. As noted above, the first part (200) of the system (100) includes a catheter assembly (300) having a proximal end and a distal end, which is configured for holding a guide wire, which is further advanced and retracted to provide a path for the entire system in a particular direction as the gastric bypass device is deployed at desired specific position.

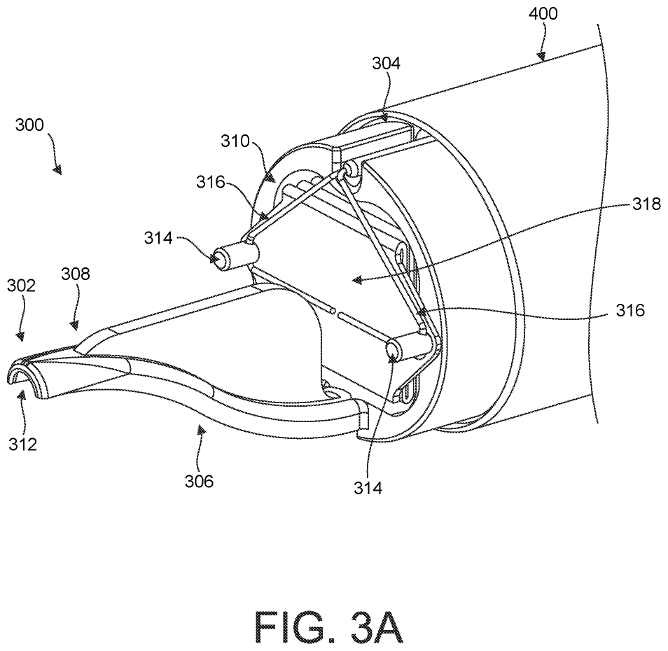

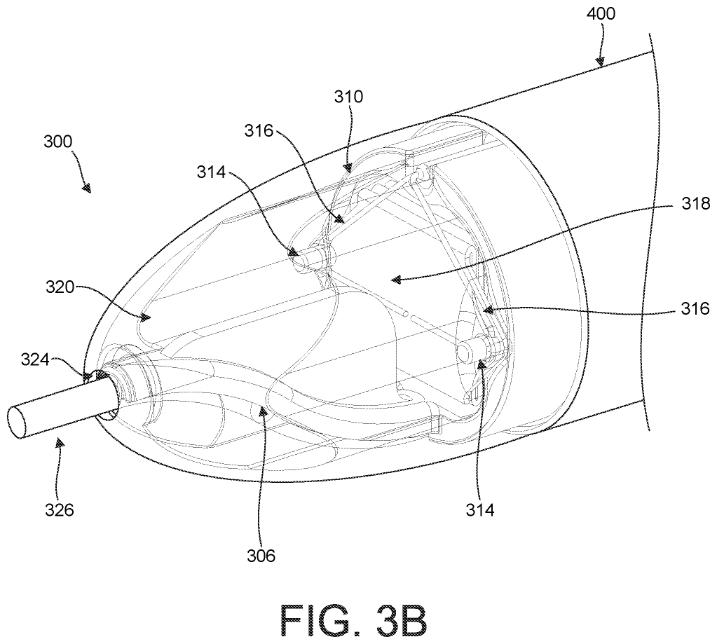

FIG. 3A depicts an interior view of the catheter assembly (300) with a distal end (302) and a proximal end (304), depicting the nose cone insert (306) in detail. The nose cone insert (306) of the catheter assembly (300) includes a front portion (308) and a back portion (310). Further, the back portion (310) of the nose cone insert (306) is connected to the capsule body (400). The front portion (308) of the nose cone insert (306) includes an opening (312), while the back portion (310) includes a pair of push wires (314) which are fitted at the diametrically opposed sides of the nose cone insert (306).

Further, the back portion (310) includes a snare wire (316), which is bifurcated in two parts and inserted with respective holes drilled within the pair of push wires (314).

The purpose of the snare wire (316) is to retain the nose cone of the catheter capsule in connection with the push wires (314) and the distal end of the sleeve (308). The snare wire (316) is routed through cross-drilled holes in the ends of the push wires (314) which protrude through locating holes in the nose cone, thus retaining the nose cone and likewise the distal end of the sleeve (308) which feature holes through which the push wires (314) were also routed in assembly. The snare wire (316) is released by withdrawing the snare wire (316) while holding the push wires (314) in position which continue to locate the nose cone at its desired distal location. The snare wire (316) is highly malleable and will withdraw from the cross-drilled holes in the ends of the push wires (314).

The back portion (310) is attached with a distal end (318) of the folded sleeve. Further, the distal end (318) of the folded sleeve is retained with the back portion (310) of the catheter assembly (300) by the engagement of the snare wire (316) with the pair of the push wires (314). The shape of the front portion (308) of the nose cone insert (306) is curved toward the opening (312). More specifically, in one aspect, the nose cone insert (306) is shaped to have a curve, similar to a nose-shape.

FIG. 3B provides an interior-view of an assembled catheter assembly (300). As shown, a nose cone (320) is fitted to the nose cone insert (306) to form an assembled catheter assembly (300), enclosing the components therein. More preferably, in one example embodiment, the nose cone (320) is snapped on the nose cone insert (306) to form the catheter assembly (300).

The proximal end of the nose cone (302) further slides with the opening (312) of the nose cone insert (306), to form a hole (324), which is configured for holding a guide wire (326). The guide wire (326) is used during operation of the device to guide placement of the gastric bypass device using endoscopy or radiology (or any other suitable technique). The guide wire (326) allows for optimal placement of the gastric bypass device. For example, during implantation, the position of the guide wire (326) is continuously monitored on the fluoroscope by means of tantalum or similar markings on the catheter assembly (300). Tantalum radiopaque markers are attached to the catheter assembly (300) at fixed increments along the length of the guide wire (326) to allow visualization of the catheter assembly (300) (along with the capsule body (400) and the capsule cover (500), further confirming the position of the implant.

The nose cone insert (306) and the nose cone (320) in this embodiment is preferably made from biodegradable or resorbable material, such as polycaprolactone, polyglecaprone, polyglactin, or other known biodegradable or resorbable materialas would be contemplated by those skilled in the art. More desirably, the biodegradable or resorbable material is polycaprolactone. The selection of the biodegradable or resorbable material is coordinated with the natural pH of the anatomical environment of the stomach, to achieve a predetermined sequential degradation of the catheter assembly (300) after being released from the expanded sleeve, thereby allowing proper implantation of the gastric bypass device within the stomach of the patient.

In one embodiment of the present invention, the guide wire (326) is used to pierce the tissue wall prior to introduction of the capsule body (400) along with the catheter assembly (300) and thereby determine if there are structures near the outer surface of the tissue that are damaged. The guide wire (326) is sufficiently flexible such that no serious bleeding or other complications would be incurred. Alternatively, the guide wire (326) can be passed through the pyloric sphincter through manipulation of the controller (700).

In one embodiment of the present invention, the pair of push wires (314), the snare wire (316), and the guide wire (326) are made up of corrosion-resistant material. More specifically, the guide wire (326) is made of a flexible material, such as steel, stainless steel or similar materials known in the art. Alternatively, the snare wire (316) is made up of two malleable thin wires. Alternatively, the snare wire (316) is made up of the annealed stainless steel or any other material known in the art.

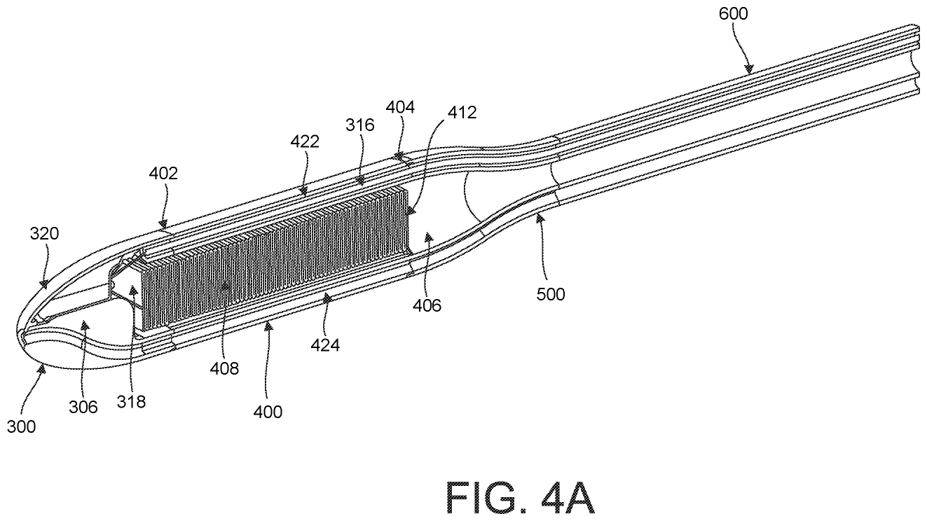

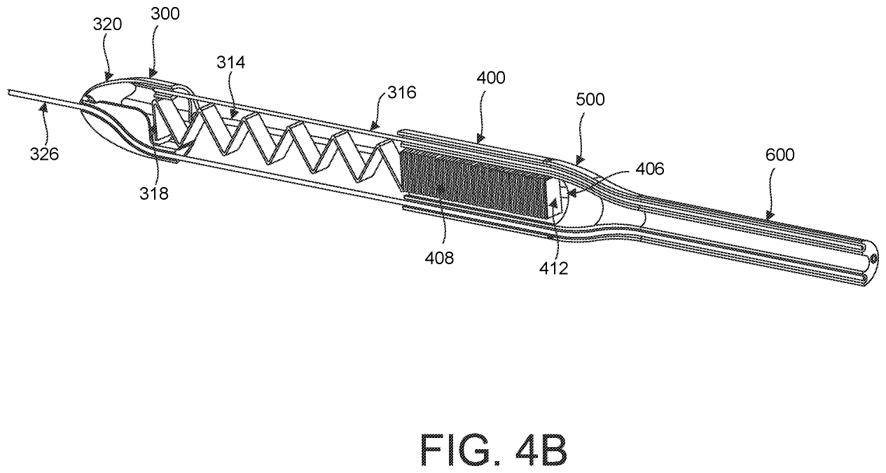

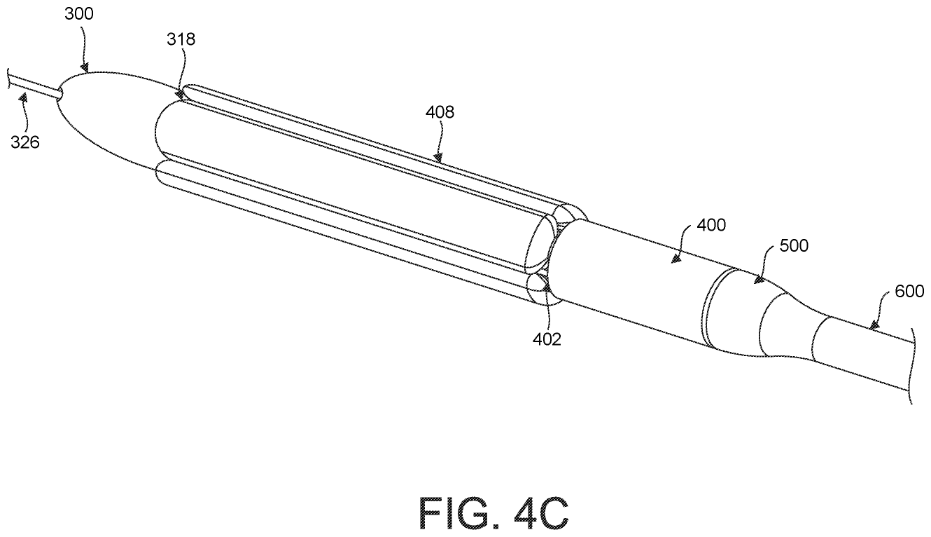

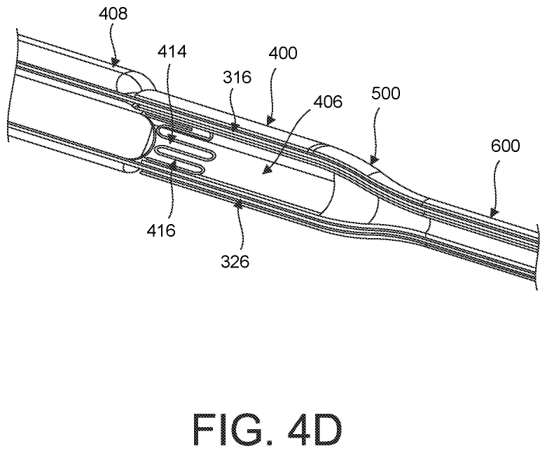

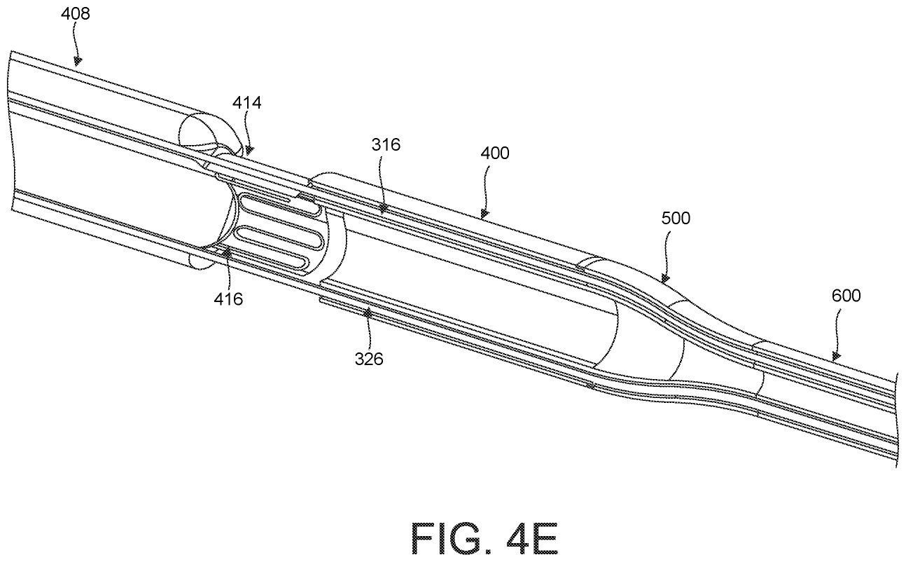

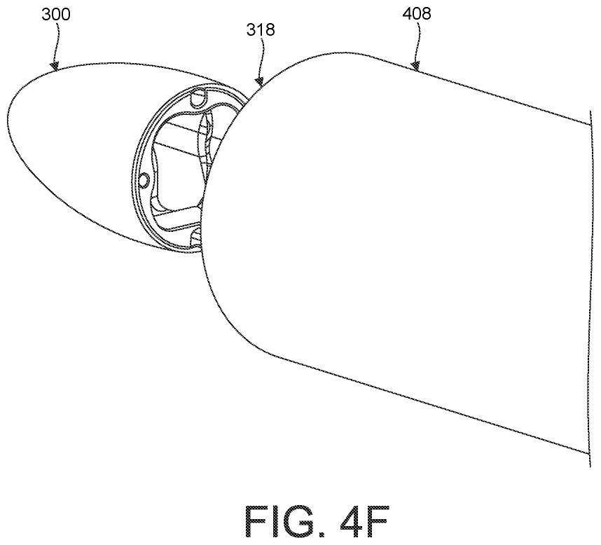

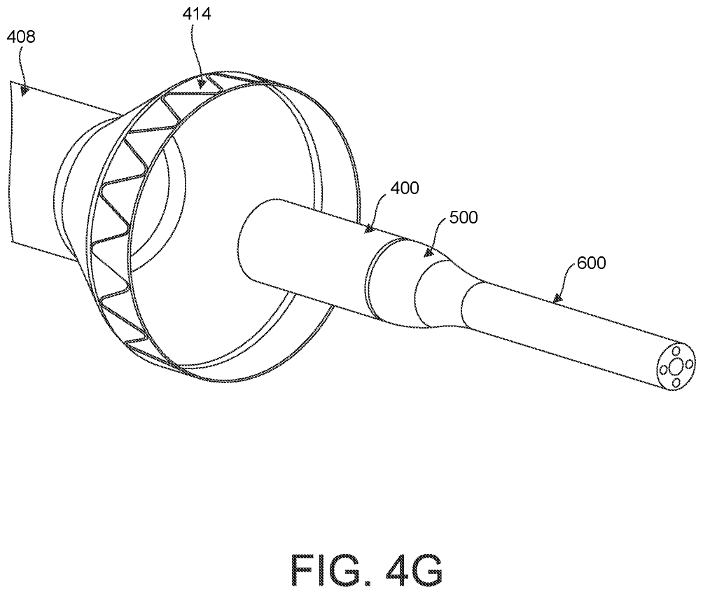

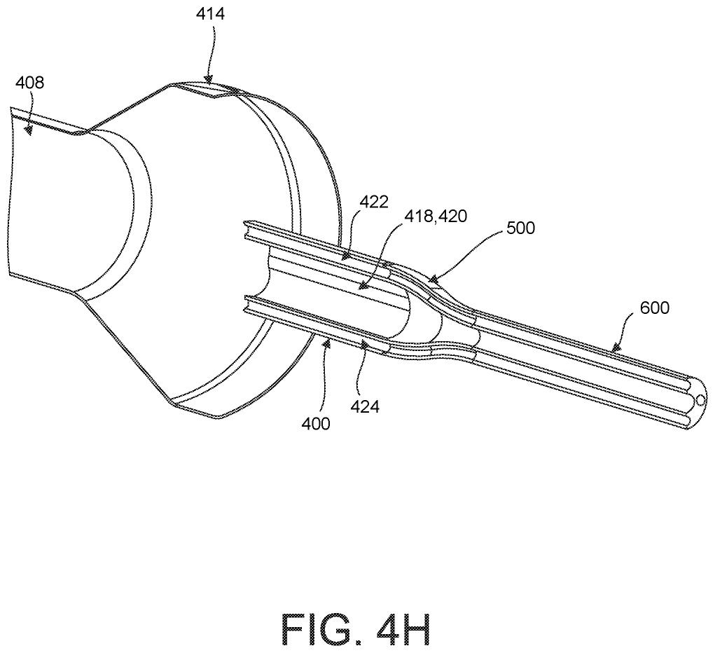

FIG. 4A represents a cross-sectional view of an assembled capsule body (400) with the catheter assembly (300) and the capsule cover (500), also depicting a portion of the flexible catheter (600). The capsule body (400) is coupled to the proximal end (304) of the catheter assembly (300). Further, the capsule body (400) includes a first end (402) and a second end (404) at opposite ends. The capsule body (400) comprises a tubular-structure that includes a hollow lumen (406) configured for holding a sleeve (408). More preferably, the sleeve (408) in one aspect is folded transversely. Thereafter and as depicted, the folded sleeve (408) is placed longitudinally within the hollow lumen (406) of the capsule body (400). More preferably, the distal end (318) of the folded sleeve (408) is affixed to the back portion of the nose cone insert (306) of the catheter assembly (300).

The folded sleeve (408) includes a front end (412) and a distal end (318). The front end (412) of the folded sleeve (408) includes the gastric bypass device (414). More specifically, the folded sleeve (408) encapsulates or fully covers the gastric bypass device (414) (shown in detail in FIG. 4E). As shown in FIG. 4E, the gastric bypass device (414) is held in place in the folded sleeve (408) by a spring wire support (416). More preferably, the spring wire support (416) is placed within internal periphery of the capsule body (400) to hold the gastric bypass device (414) with the front end (412) of the folded sleeve (408). The spring wire support (416) is made of any suitable material, such as a nitinol material or similar material as would be contemplated by a person skilled in the art.

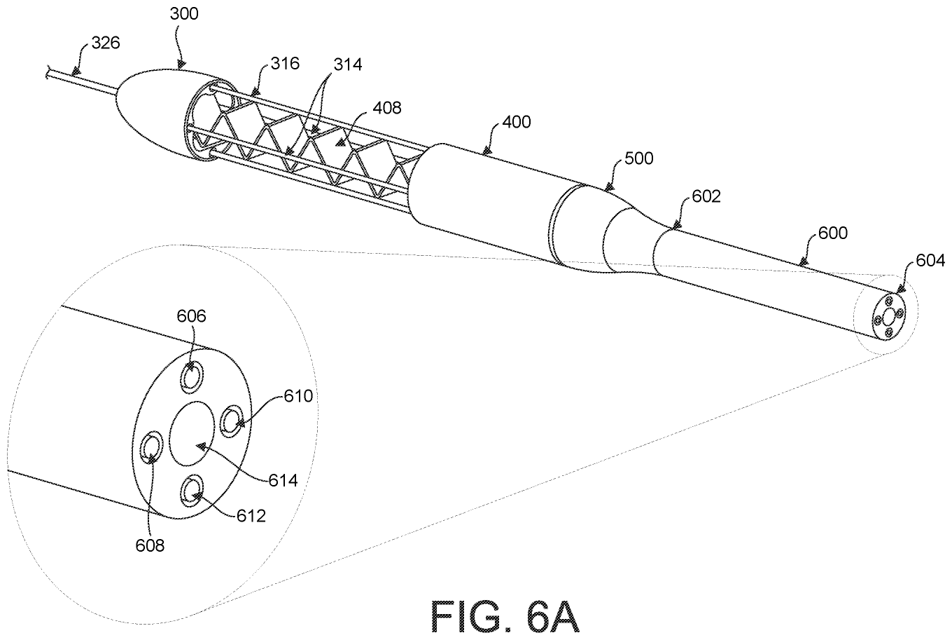

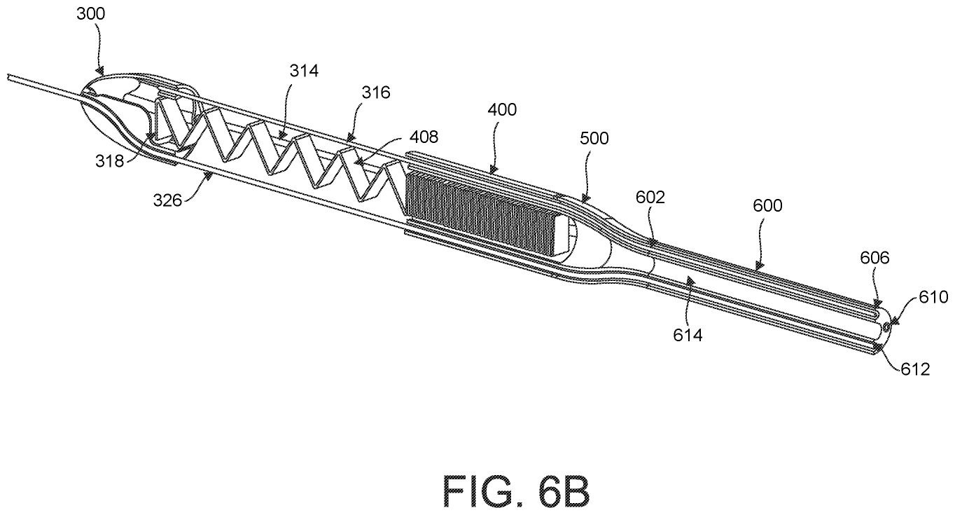

As shown in FIGS. 4E and 4H, the capsule body (400) further includes multiple channels (418, 420, 422, 424) longitudinally arranged around the folded sleeve (408) to place each of the pair of the push wires (314), the snare wire (316), and the guide wire (326) coming from the flexible catheter (600) to the first end (402) of the capsule body (400). More preferably, the capsule body (400) includes four channels (418, 420, 422, 424) arranged longitudinally around the folded sleeve (408) that align with the holes (depicted in FIG. 6A as 606, 608, 610, 612) formed through the flexible catheter (600). A pair of the channels (418, 420) are formed at opposing sides to hold the pair of the push wires (314) as received from corresponding holes in the flexible catheter (600). A top channel (422) is arranged at a topmost end to hold the snare wire (316) as received from the flexible catheter (600). Further, the capsule body (400) includes a bottom channel (424) arranged at a lowermost end to hold the guide wire (326) as received from the flexible catheter (600). More specifically and as shown between FIGS. 3B and 4A, the snare wire (316) and pair of the push wires (314) converge at the first end (402) of the capsule body (400) and the back portion (308) of the nose cone insert (306) of the catheter assembly (300). Further, the snare wires (316) along with the pair of the push wires (314) hold the distal end (318) of the folded sleeve (408).

In one aspect, the capsule body (400), the folded sleeve (408) and the gastric bypass device (414) are made of any suitable material, non-limiting examples of which include polytetrafluoroethylene (PTFE), polyethylene, cast PTFE, Teflon, fluorinated ethylene propylene (FEP), perfluoroalkoxy (PFA), PTFE, PFA, extruded FEP and extruded PFA, extruded PTFE, and silicone. More preferably, the folded sleeve (408) and the gastric bypass device (414) are made of polyethylene or similar material known in the art. The device with the different components and the tube may be made of plastic, polyethylene and other moldable material as would be contemplated by the person skilled in the art.

FIG. 4B provides a cross-sectional view of the capsule body (400) coupled to the proximal end (304) of the catheter assembly (300), as shown in FIG. 3A. The capsule body (400) is further connected with the capsule cover (500) with a portion of the flexible catheter (600). During operation, the capsule body (400) is slowly separated from the catheter assembly (300) by withdrawing the flexible catheter (600) by means of the controller (700). More specifically, the capsule body (400) is withdrawn along with the capsule cover (500) and the flexible catheter (600) by advancing the pair of the push wires (314) and the snare wire (316) by means of the controller (700). The pair of push wires (314) are held in position while withdrawing the capsule body (400) from the catheter assembly (300).

As the capsule body (400) is withdrawn, along with the flexible catheter (600) while maintaining the position of the catheter assembly (300), the folded sleeve (408) starts unfolding from the capsule body (400) while maintaining the distal end (318) of the folded sleeve (408) that is still connected to the catheter assembly (300). More specifically, the capsule body (400) is withdrawn from the catheter assembly (300) to a desired position in the duodenum when the folded sleeve (408) is partially unfolded and in a partially folded state.

To deploy the gastric bypass device (414) in the small intestine (i.e., antrum proximal region of the pyloric sphincter), the capsule body (400) is deployed. The capsule body (400) is deployed to the desired location in the small intestine along the previously deployed guide wire (326). The location of both the capsule body (400) along with the catheter assembly (300) is then monitored on a fluoroscope by means of tantalum or similar markings.

FIG. 4C represents a perspective view of the capsule body (400) from the catheter assembly (300), showing the inflated sleeve (408) from the proximal end (304) of the catheter assembly (300) and the first end (402) of the capsule body (400), as shown in FIG. 3A. In use, the capsule body (400) is positioned to the desired location and verified through fluoroscopy. Upon verifying the position of the capsule body (400), the unfolded sleeve (408) is provided with a fluid to complete the unfolding of the folded sleeve (408). Fluid is introduced to the unfolded sleeve (408) through the flexible catheter (600) to assist in the complete unfolding of the fully unfolded sleeve (408). Thus, by introducing the fluid, an inflated sleeve (408) formed.

The fluid introduced is either gas or a liquid. The gas may be air, carbon dioxide gas or similar gas known in the art. The liquid may be saline solution, water or similar solution known in the art. In one desired aspect, a saline solution is introduced through the flexible catheter (600) to assist in the complete unfolding of the unfolded sleeve (408).

Once the partially unfolded sleeve (408) is fully unfolded, the distal end (318) of the unfolded sleeve (408) is still attached to the back portion (310) of the nose cone insert (306) through the engagement of the snare wire (316) and the pair of the push wires (314). The front end (412) of the unfolded sleeve (408) remains affixed within the hollow lumen (406) of the capsule body (402) and is further withdrawn to release the gastric bypass device (414), as shown in FIG. 4B and FIG. 4D. The snare wire (316), the push wires (314) and the guide wire (326) are all held at their respective positions as shown in FIG. 4B. More specifically, the front end (412) of the inflated sleeve (408) is connected with the first end (402) of the capsule body (400), and the distal end (318) of the inflated sleeve (408) is connected with the proximal end (304) of the catheter assembly (300), as shown in FIG. 3A.

FIG. 4D provides a cross-sectional view of the inflated sleeve (408), depicting the gastric bypass device (414) as held by a spring wire support (416) within the hollow lumen (406) of the capsule body (400). More specifically, the spring wire support (416) holds the gastric bypass device (414) within the internal periphery of the hollow lumen (406) of the capsule body (400). The spring wire support (416) can be made in any suitable shape. As a non-limiting example, the spring wire support (416) is in a serpentine shape. Thus, in one aspect, the gastric bypass device (414) remains in the hollow lumen (406) of the capsule body (400), folded back on itself and retained by the serpentine spring wire support (416) pressing against the inner surface of the capsule body (400).

Further and as shown in FIG. 4D, the capsule body (400) is slowly withdrawn from the catheter assembly (300) by advancing the pairs of the push wires (314) and the snare wire (316). All the push wires (314), the snare wire (316) and the guide wire (326) are held in position and further advanced by the controller (700) to maintain the position of the catheter assembly (300).

As the capsule body (400) is further withdrawn, the entire inflated sleeve (408) comes out of the capsule body (400), with the exception of the portion that includes the gastric bypass device (414). At this point the gastric bypass device (414) is held in position by the compressed wire support (416). All the push wires (314), the snare wire (316) and the guide wire (326) remain in place and are advanced by the controller (700) to maintain the position of the catheter assembly (300), which is monitored continuously through fluoroscopy during this process.

FIG. 4E represents a cross-sectional view of the inflated sleeve (408) after being removed from the capsule body (400), showing emergence of the gastric bypass device (414) with the spring wire support (416). The capsule body (400) is further withdrawn by advancing the pair of push wires (314) and the snare wire (316). The gastric bypass device (414) along with the spring wire support (416) emerges outside from the capsule body (400), along with the inflated sleeve (408). All the push wires (314), the snare wire (316) and the guide wire (326) remain in place and are advanced by the controller (700) to maintain the position of the catheter assembly (300), which is monitored continuously through fluoroscopy during this process.

FIG. 4F represents a perspective view of the release of the catheter assembly (300) from the distal end (318) of the inflated sleeve (408). Given the description above, it should be understood that the guide wire (326) is removed from the capsule body (400) by means of the controller (700). Further, the pair of push wires (314) and snare wire (316) are also retracted from the capsule body (400) using the controller (700). At this point, the front end (412) of the inflated sleeve (408) is still held by the spring wire support (416). The catheter assembly (300) is then released from the distal end (318) of the sleeve (408) to release the inflated sleeve (408) within the duodenum of the patient. The distal end (318) of the inflated sleeve (408) is set free within the duodenum of the patient.

FIG. 4G represents a view of the funnel-shaped gastric bypass device (414). Desirably, the gastric bypass device (414) is conical, funnel or similar shape. The funnel shape conforms to the lower end of the stomach cavity in the antrum, which helps induce satiety and the inflated sleeve (408) continues and widens in a proximal direction below the funnel shaped gastric bypass device (414). Once positioned, the funnel shaped gastric bypass device (414) is secured to the patient's tissue using the support wire spring (416) as reinforcement. The front end of the funnel shaped gastric bypass device (414) is then sutured to the gastric wall just above the previously placed suture. More specifically, the cone shaped or funnel shaped gastric bypass device (414) is deployed in the antrum, proximal to the pyloric sphincter.

Once deployed, multiple interrupted sutures or continuous suture can be placed. The front end of the funnel shaped gastric bypass device (414) is provided antrum circumferential sutures using any suitable overstitching process. As a non-limiting example, the overstitches are applied using the Apollo overstitch processor device as provided by Apollo Endosurgery, Inc. located 1120 S Capital of Texas Hwy., Bldg 1, Suite 300, Austin, Tex. 78746 USA. Alternatively, interrupted sutures can be placed using the Apollo overstitch stitching device. It should be noted that attention must be paid during placement of the sutures to ensure that the proximal end of the funnel is at the level of the circumferential suture, placed at the junction of fundus and antrum.

In another aspect, the gastric bypass device (414) is formed to have a conical, funnel, or frusto-conical shape, and is deployed in the antrum, proximal to the pyloric sphincter. In this aspect, the funnel shaped gastric bypass device (414) is placed in the stomach. Further, the inflated sleeve (408) extends from the funnel shaped gastric bypass device (414) to the pylorus and is semi flexible in nature. Thus, the inflated sleeve (408) opens in the small bowel and traverses the entire small bowel to the jejunum, creating the duodeno-jejunal barrier. Upon placement in the stomach, the gastric bypass device (414) bypasses part of the stomach and acts as a sheath like barrier to cover the duodenum and part of the jejunum.

The inflated sleeve (408) may include a length of, e.g., approximately 100 cm long, or any other desired length. Alternatively, the inflated sleeve (408) may be longer than 100 cm. In yet another aspect, the inflated sleeve (408) may be shorter than 100 cm. In one embodiment of the present invention, the gastric bypass device (414) is a squeezed funnel which measures approximately 5 cm in length. Alternatively, the funnel shaped gastric bypass device (414) can be more than 5 cm.

In one embodiment of the present invention, the inflated sleeve (408) with the gastric bypass device (414) is formed of a material that retains structural integrity sufficiently for a desired time period to perform its intended function within the stomach of the patient, and then dissolves. Thus, in this aspect, the components are biodegradable. Alternatively, the inflated sleeve (408) with the gastric bypass device (414) is non-biodegradable for the desired time-period.

More specifically, the capsule body (400), the capsule cover (500) and the gastric bypass device (414) along with sleeve (408) are made of any one of the polytetrafluoroethylene (PTFE), polyethylene, cast PTFE (e.g. Teflon), fluorinated ethylene propylene (FEP), perfluoroalkoxy (PFA), PTFE, PFA, extruded FEP and extruded PFA, extruded PTFE, silicone. More preferably, the folded sleeve (408) and the gastric bypass device (414) of the present application is made of polyethylene. Alternatively, the folded sleeve (408) and the gastric bypass device (414) may be made of plastic, polyethylene and other moldable material.

The gastric bypass device (414) is to be made of a compliant plastic material. Due to this reason, no piercing of the stomach or intestine walls is required to fix this device within the stomach of the patient. Thus, the gastric bypass device (414) is implanted using a minimally invasive method that does not require any surgical procedure (e.g., through the esophagus). The gastric bypass device (414) can then be removed at any time without any further tissue modifications.

For further understanding, FIG. 4H represents a cross-sectional view of the fully expanded gastric bypass device (414) along with the inflated sleeve (408) as deployed within the stomach of the patient. The catheter assembly (300), the flexible catheter (500) along with the capsule body (400) and the capsule cover (600) are then withdrawn from the stomach. The front end (412) of the inflated sleeve (408) is released by removal of the spring wire support (416), thereby deploying the gastric bypass device (414) in the antrum proximal to the pyloric sphincter. Alternatively, the gastric bypass device (414) is deployed within the mid-region of the stomach.

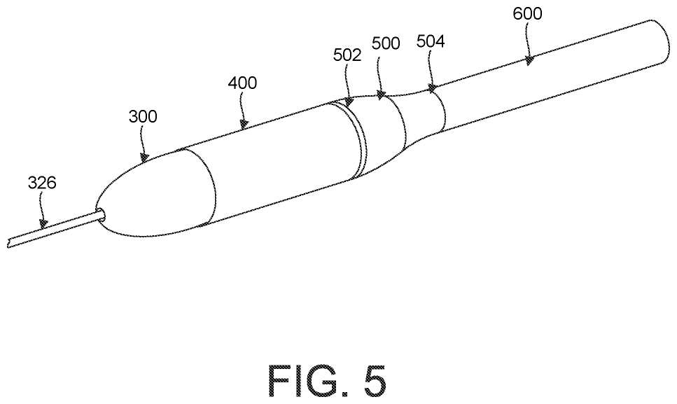

For completeness, FIG. 5 depicts the capsule cover (500) connected with the capsule body (400). In other words, the capsule cover (500) is connected in between the capsule body (400) and the flexible catheter (600). The capsule cover (500) is connected to the second end of the capsule body (400) through the one end (502) and is connected to the flexible catheter (600) through another end (504). More specifically, the capsule cover (500) may be snap fit with the capsule body (400) to form a single structure deployed via the flexible catheter (600) in the esophagus of the patient, with the gastric bypass device (414) then implanted within the stomach at desired position. The capsule cover (500) is made of any desired material, such as the same material as the capsule body (400). The material is selected from any one of the polytetrafluoroethylene (PTFE), polyethylene, cast PTFE (e.g., Teflon), fluorinated ethylene propylene (FEP), perfluoroalkoxy (PFA), PTFE, PFA, extruded FEP and extruded PFA, extruded PTFE, and silicone.

As noted above, the delivery device also includes a flexible catheter (600). As shown in FIGS. 6A and 6B, the flexible catheter (600) is connected with capsule body (400) via capsule cover (500). The flexible catheter (600) is connected to another end of the capsule cover (500). In one embodiment, a fluid-tight seal is created between the another end of the capsule cover (500) and the one end (602) of the flexible catheter (600) (as shown in FIG. 5). Further, the flexible catheter (600) includes multiple holes (606, 608, 610, 612) and a central lumen (614) extending between the lumen of the capsule cover (500) and the bottom end (604) of the catheter (600).

As shown on the bottom end of the flexible catheter (600), a first hole (606) is formed through the catheter (600) to receive a snare wire (316), which is further received by the channel formed in the first end (402) of the capsule body (400). The flexible catheter (600) further includes a pair of second holes (608, 610) at opposing sides to receive push wires (314) from each of the pair of second holes (608, 610). Each of the push wires (314) are then received in the corresponding channels formed at the first end (402) of the capsule body (400). The flexible catheter (600) also includes a hole (612) at a bottom portion to receive the guide wire (326), which is thereafter received at the hole of the proximal end (302) of the catheter assembly (300) via the capsule body (400) and the capsule cover (500).

A central lumen (614) is provided through the flexible catheter (600) for transferring a fluid to the folded sleeve (408) within the capsule body (400) via the capsule cover (500). More desirably, the flexible catheter (600) is in fluid communication with the controller (700), which flushes the flexible catheter (600) with inflation fluid. In some embodiment, an endoscope can flush the flexible catheter (600) with a fluid source. More desirably, the flexible catheter (600) is made of silicone or any other materials as would be contemplated by those skilled in the art to prevent degradation by acidic gastric contents within the stomach.

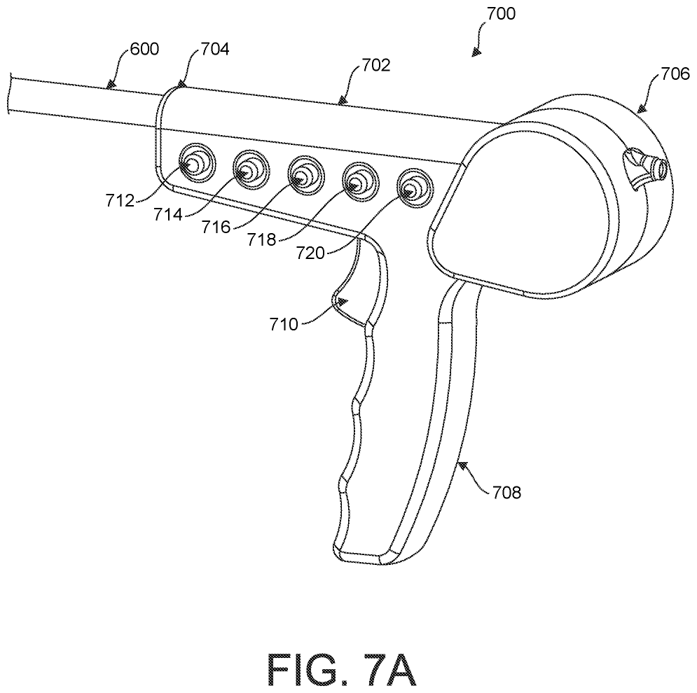

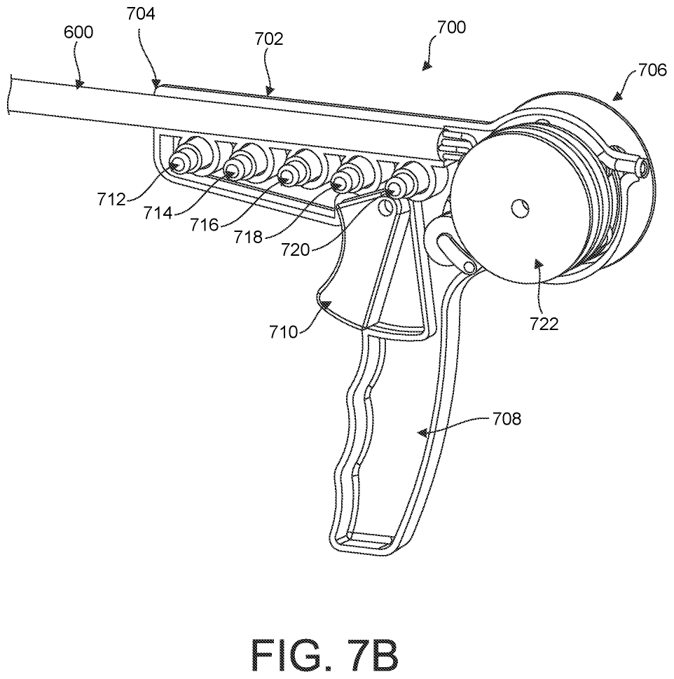

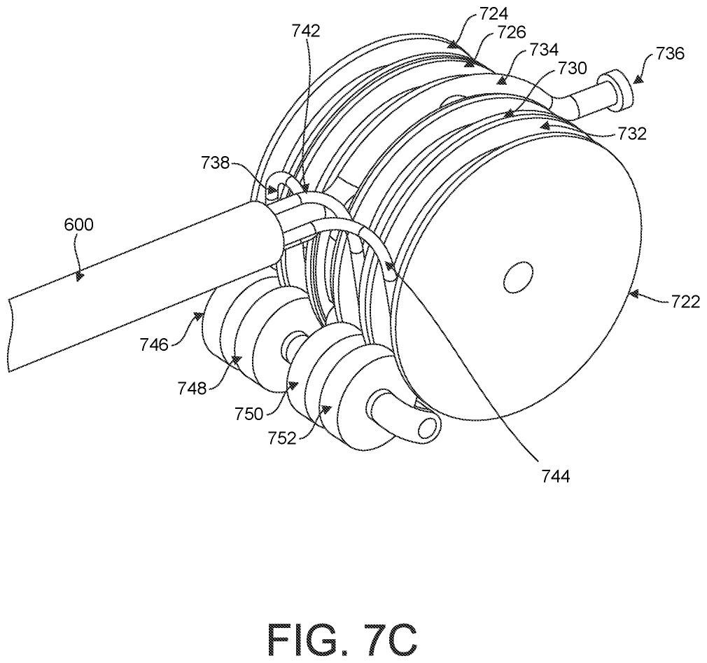

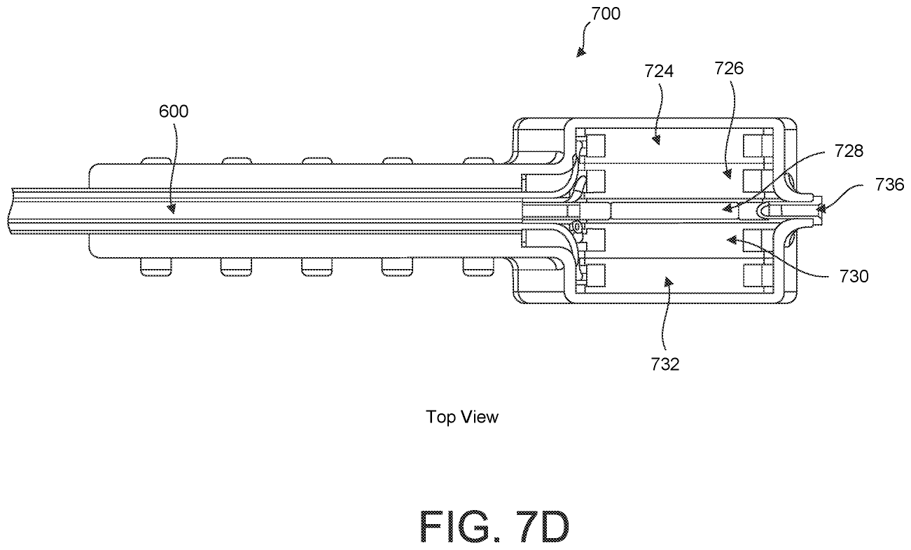

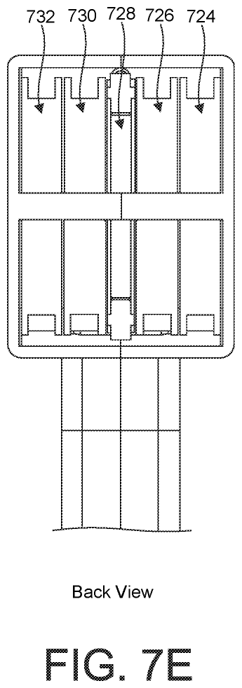

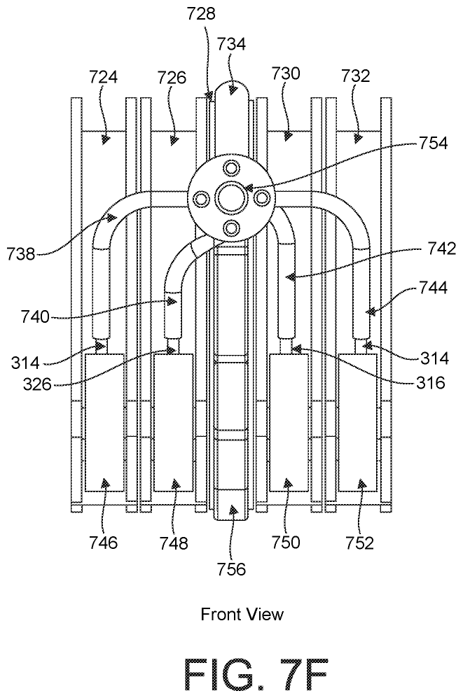

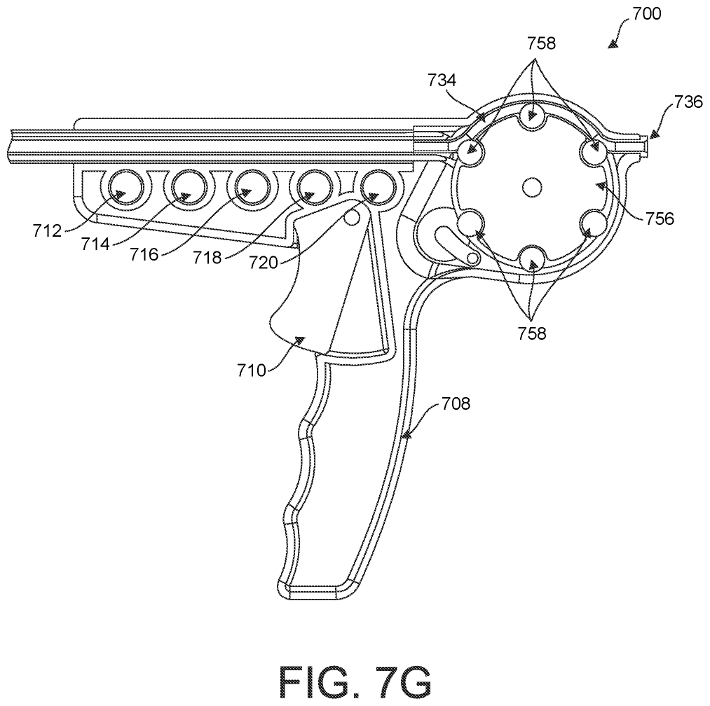

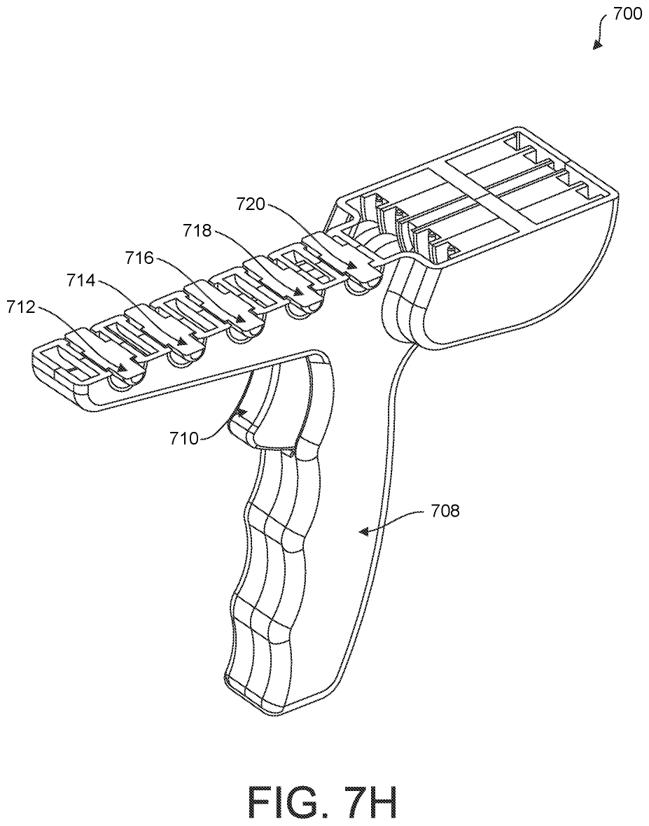

FIG. 7A depicts the controller (700) as coupled to the flexible catheter (600) in a detachable configuration. The controller (700) is configured for providing controlling commands for precise advancement and retraction of the guide wire (326), the pair of push wires (314) and the snare wire (316). Further, the controller (700) also provides commands for transferring fluid in the sleeve (408) via the capsule body (400) and the flexible catheter (600). In one embodiment of the present invention, the controller (700) may be an automatic or a manual controller. In another aspect, the controller (700) is an electrically controlled electrical device.

More preferably, the controller (700) is a hand-held, manually controlled controller. Although not limited thereto, in a desired embodiment, the controller (700) is shaped like a handgun to allow for comfort and ease of control. In this aspect, the controller (700) includes a gun-shaped member (702) with a distal end (704) and a proximal end (706). The gun-shaped member (702) receives the flexible catheter (600) from the distal end (704) towards the proximal end (706) of the gun-shaped member (702). The controller (700) also includes a hand-grip member (708) that is configured to be griped by the operator, surgeon or any person as would be contemplated by those skilled in the art.

The controller further includes a trigger (710) for initiating action based on selection of the operation to be performed. The action is either precise advancement or retraction of one of the guide wire (326), the snare wire (316) or the pair of push wires (314), or the transfer of fluid from the fluid transfer tube (734) as shown in FIG. 7F. In one embodiment of the present invention, the trigger (710) is either an actuator lever or a hand crank knob. More desirably, the action is performed by pressing the trigger (710). In another aspect, the controller (700) can be formed to employ a hand crank or turn wheel on the side of the controller (700) aligned with the wire axis spool.

To allow for multiple control options, the controller (700) includes multiple function selectors (712, 714, 716, 718, 720) that are linearly arranged at the distdal end (704) of the gun-shaped member (702). Although not limited thereto, in one aspect, the multiple function selectors (712, 714, 716, 718, 720) are placed below the gun shaped member (702) holding the flexible catheter (600). The multiple function selectors (712, 714, 716, 718, 720) operate as mode selectors to allow a user to select operation of snare wire (316) engagement, push wire (314) engagement, guide wire (326) engagement, fluid transfer engagement (via the fluid transfer tube (734)), and forward/reverse selector (as shown in detail in FIG. 7E), respectively.

The function selectors (712, 714, 716, 718, 720) can be formed in any suitable manner. For example, they can be either buttons or turn knobs. More preferably, the function selectors (712, 714, 716, 718, 720) are buttons that are pressed for selecting operations to be performed with the various wires and the transfer of fluid.

FIG. 7B provides a cross-sectional view of the controller (700), depicting a spooler (722) mechanism that includes multiple spools configured for holding multiple wires (i.e., the guide wire (326), the pair of push wires (314) and the snare wire (316), and the fluid transfer tube (734)). For further understanding, FIG. 7C provides a perspective view of the spooler mechanism (722), showing the various spools with the wires thereon. Specifically, the spooler mechanism includes a push wire spool (724), a guide wire spool (726), a peristaltic pump, a snare wire spool (730), and second push wire spool (732) with channels for controlling corresponding wires. Further, the spooler (722) also includes a peristaltic pump with a channel (728) (as shown in FIG. 7D) for holding a fluid transfer tube (734) that is fluidly connected to a fluid inlet (736) for introduction of fluid to the device. The fluid inlet (734) is provided at rear end of the spooler (722) and is further connected to a fluid source. In one embodiment of the present invention, the fluid source is integrated with an endoscope for transferring the fluid to the capsule body (400) via the capsule cover (500) and the flexible catheter (600).

The spooler (722) also includes multiple wire guiding tubes (738, 740, 742, 744) that are placed on top of the multiple channels of each of the spools. The multiple wire-guiding tubes (738, 740, 742, 744) are operable for receiving each of the guide wire (326), the snare wire (316) and the pair of push wires (314) from the flexible catheter (600) and guiding the relevant wires onto the corresponding channels of the relevant spools (724, 726, 730, 732) of the spooler (722). The spooler (722) also includes multiple contra-rotating friction rollers (746, 748, 750, 752) placed at the bottom of each of the spools (724, 726, 730, 732) for feeding or advancing the wires to and from the respective spools (724, 726, 730, 732). More specifically, depending upon which button is depressed, the contra-rotating friction rollers (746, 748, 750, 752) are pressed against the wires of the spooler (722) to advance the guide wire (326), the snare wire (316) and a pair of push wires (314) into the wire guiding tubes (738, 740, 742, 744), respectively. Alternatively, when in the retraction mode, the contra-rotating friction rollers (746, 748, 750, 752) are released from the corresponding spool on the spooler (722) to retract the relevant guide wire (326), snare wire (316) or push wires (314).

As can be understood by those skilled in the art, the function selectors (712, 714, 716, 718, 720) include any mechanism or parts as may be required to allow for selective control of the friction rollers (746, 748, 750, 752) and corresponding spools (724, 726, 730, 732) or pump as applicable. As a non-limiting example, a set of concentric shafts are individually rotated by a small degree by pressing a button on one side of the controller (700). These shafts would be rotated back to their initial position by depressing the other end of the button, which has now protruded on the other side of the controller (700). The shaft rotation is indexed to remain in one of two angular positions corresponding to the position of the buttons running crosswise through the controller (700). The shafts would actuate individual brakes for each friction roller (746, 748, 750, 752) or pump, through a mechanism, such as cams or rod linkages. The purpose of the friction rollers (746, 748, 750, 752) is to be able to play out wire through the formed tubes which lead to the catheter as well as lock the position of a wire. In one non-limiting example, the wire spools (724, 726, 730, 732) are spring-loaded with a clock spring to take up wire when the brakes on the friction rollers (746, 748, 750, 752) are released (e.g., through use of the trigger). An additional force can also be added to withdraw wires which. For example, a hand crank(s) or a motor(s) can be included to provide the required torque to withdraw the wires. In addition, there is a need for torque to be applied to the friction rollers (746, 748, 750, 752) to play out the guide wire or other wires before insertion of the catheter at the beginning of the procedure and to hold the catheter assembly in position with the push wires when withdrawing the catheter.

For further understanding, FIGS. 7D and 7E illustrates a top, cross-sectional view of the flexible catheter (600) and cross-sectional back view of the spooler (722), respectively.

Further, FIG. 7F represents a front view of the spooler (722), detailing the spools (724, 726, 730, 732) having the guide wire (326), the snare wire (316) and the pair of push wires (314). Further and as shown, the spooler (722) includes the fluid inlet (736) and the fluid transfer tube (734). Also as shown, each of the guide wire (326), the snare wire (316) and the pair of the push wires (314) are fed into the respective wire guiding tubes (738, 740, 742, 744) through respective channels of the corresponding spools (724, 726, 730, 732). Each of the wire guiding tubes (738, 740, 742, 744) are connected at common junction (754) for directing the relevant wires and fluid transfer tube (734) into the flexible catheter (600).

FIG. 7G provides a cross-sectional view of the hand-held controller (700) in a fluid-transfer mode, showing transfer of the fluid from the fluid transfer tube (734) through engagement of the peristaltic pump (756) with multiple rollers (758). The fluid transfer from the fluid transfer tube (734) is controlled from a peristaltic pump rotor (756) placed in the channel (728) below the fluid transfer tube (734). The multiple rollers (756) placed at a periphery of the peristaltic pump rotor (754) regulate the transfer of fluid from the fluid transfer tube (734).

As the fluid transfer mode is engaged by pressing the fluid transfer button (718), the peristaltic pump rotor (754) rotates the multiple rollers (758) for transferring fluid to the fluid transfer tube (734). Once the sleeve (408) is fully unfolded, the transfer of the fluid to the capsule body (400) is terminated. The transfer of fluid leads to the expansion of the unfolded sleeve (408) to form the inflated sleeve (408) followed by the disengagement of the capsule body (400) from the inflated sleeve (408) and further deployment of the gastric bypass device (414) within the stomach of the patient.

The fluid is either a gas or a liquid. The gas may be air, carbon dioxide any other gas known to person skilled in the art. The liquid may be saline solution, water or any other liquid known to person skilled in the art. More specifically, the saline solution introduced through a central lumen (612) in the flexible catheter (600) to assist in the complete unfolding of the flexible sleeve (408).

As shown in FIG. 7H, from the proximal end of the gun shaped member, the first button (712) is for engaging the snare wire (316), performed by depressing the button (712) in one direction, which once engaged, allows for advanced or retracted operation of the snare wire (316) by pressing the trigger (710). Alternatively, the button (712) can be depressed from the opposite direction to disengage the functioning of the snare wire (316).

Similarly, the second button (714) is for engaging the pair of push wires (314), performed by depressing the button (714) in one direction which then allows for advancement or retraction using the trigger (710). Alternatively, the button (714) can be depressed from the opposite direction to disengage the functioning of the pair of the push wires (314).

The third button (716) is for engaging the guide wire (326), performed in a similar manner as that described above where depressing the button (716) in one direction allows for engagement while pressing the button (716) from the opposite direction disengages the functioning of the guide wire (326).

The fourth button (718) is for engaging the fluid transfer mode, performed by depressing the button (718) in one direction and then the button (718) can be depressed from the opposite side to disengage the fluid transfer mode.

The fifth button (720) is for mode selection allowing the user to select to either advance or retract the wires. Thus, the fifth button (720) is for engaging either the advance mode or retraction mode. The fifth mode selection button (720) is depressed from one side to engage the advancement function (i.e., advancement of the selected wires). The mode selection button (720) is depressed from other side to engage the retraction function (i.e., retraction of the selected wires).

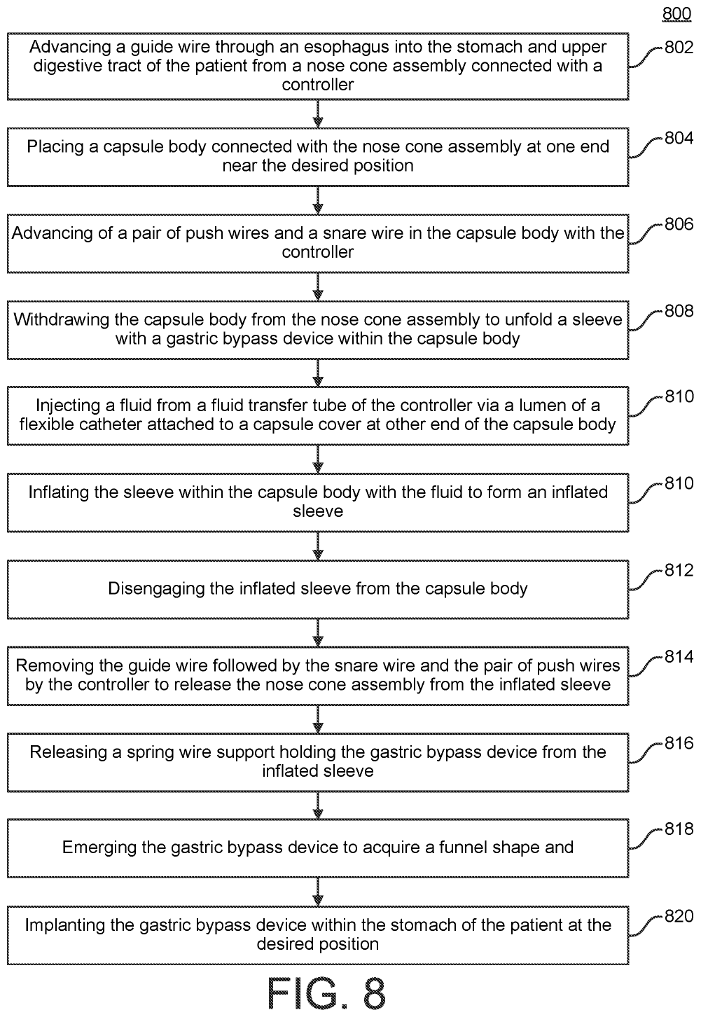

As noted above and as shown in FIG. 8, the present disclosure also provides a method for implanting a gastric bypass device (414) within a stomach of a patient. The method includes a series of steps. For example, the process is initiated at 802 by engaging a guide wire (326) transfer mode through the controller (700). In this step, the guide wire (326) is advanced through an esophagus into the stomach and upper digestive tract of the patient from a catheter assembly (300) at the desired position.

At step 804, a capsule body (400) with a catheter assembly (300) is positioned near the desired position by deploying the capsule body (400) over the guide wire (326).

At step 806, by engaging a push wire (314) transfer mode through the controller (700), a pair of push wires (314) is advanced. Further, by engaging a snare wire (316) transfer mode through the controller (700), a snare wire (316) is advanced within the capsule body (300).

At step 808, the capsule body (400) is further withdrawn from the catheter assembly (300) to unfold a sleeve (408) with a gastric bypass device (414) within the capsule body (400).

At step 810, a fluid is injected from a fluid transfer tube (736) of the controller (700) via a lumen (614) of a flexible catheter (600).

At step 812, the sleeve (408) is inflated within the capsule body (400) with the fluid to form an inflated sleeve (408).

At step 814, the inflated sleeve (408) is disengaged from the capsule body (400) by further withdrawing the capsule body (400).

At step 816, the guide wire (326) is removed by engaging the guide wire (326) mode and further retracting the guide wire (326) by the controller (700). Further, the method includes removing the snare wire (316) by engaging the snare wire (316) mode and further retracting the snare wire (316) by the controller (700). Furthermore, this process is followed by removing the pair of push wires (314) by engaging the push wire (314) mode and retracting the push wire (316) with the controller (700) in order to release the catheter assembly (300) from the inflated sleeve (408). Furthermore, it is followed by releasing the catheter assembly (300) from the capsule body (400).

At step 818, a spring wire support (416) is released by holding the gastric bypass device (414) from the inflated sleeve (408). The spring wire support (416) is released from the catheter capsule by continuing to withdraw the catheter after positioning and holding the nose cone at the distal position by playing out the two “push” wires at the same rate as withdrawing the catheter.

At step 820, the gastric bypass device (414) emerges to acquire a funnel shape.

At step 822, the gastric bypass device (414) is implanted within the stomach of the patient at the desired position.

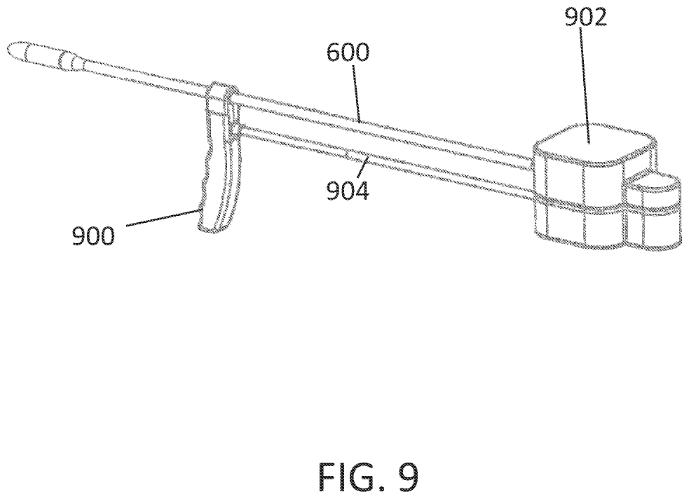

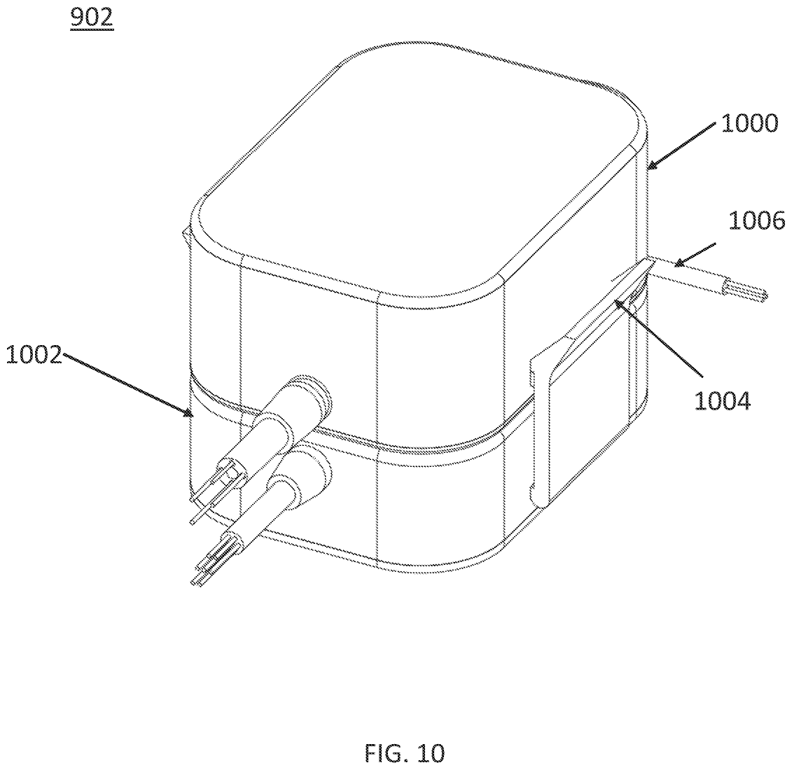

It should be noted that the description as provided above with respect to the controller (700) is directed to one example embodiment and that other versions of the controller can be implemented according to various embodiments of the present invention. For example, FIGS. 9 through 17 depict an example in which the controller is automated or motorized. In this example, the control handle (900) has electronic switches and components that could either be disposable or reusable. In one example embodiment, the spools are each operable by a motor or other electronically actuated mechanism or device that is housed within a base station. For example and as shown in FIG. 9, the control handle (900) holds the flexible catheter (600) and also holds an electrical cable (904) that is used to control all of the motors and spools that are housed within a base station (902), which in turn allows for control of all the relevant wires within the flexible catheter (600). In one aspect, the control handle (900) and its cable (904) could be connected via a bulkhead connector to allow replacement if required. For further understanding, FIGS. 10 through 17 illustrate the base station (902) and its interior components, including the spools and motors, a pumping mechanism, etc. Thus, as can be appreciated by those skilled in the art, the electronic version includes all of the necessary components as may be necessary to allow for motorized and/or automated control of the catheter assembly as described above and herein.

For example and as shown in FIG. 10, the gastric bypass device can be formed in an automated version in which the base station (902) has a bespoke single-use disposable component (1000) mounted atop a reusable base component (1002). The disposable component 1000 mounts onto the reusable base component (1002) using any suitable mechanism or technique in which a user can selectively detach/attach the components with respect to one another, a non-limiting example of which includes side-mounted latches (1004). The reusable base component (1002) is powered by battery or any other suitable power source, such as a wall main AC line (1006), either directly or via an external DC convertor power supply.

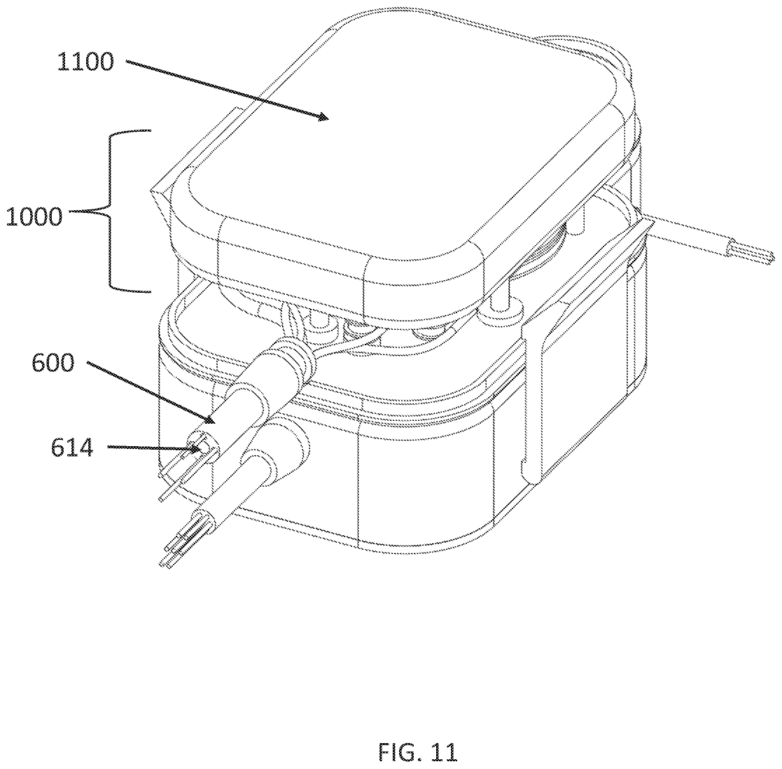

As shown in the interior-view of FIG. 11, the disposable component (1000) includes a bladder (1100) for flushing fluid through the lumen (614) in the catheter (600) and fluid transfer is accomplished by means of a peristaltic pump (or other pumping means) featured in the disposable component (1000). In one aspect, the disposable component (1000) is integrated with the catheter (600) and capsule body (400) for ease of use and deployment.

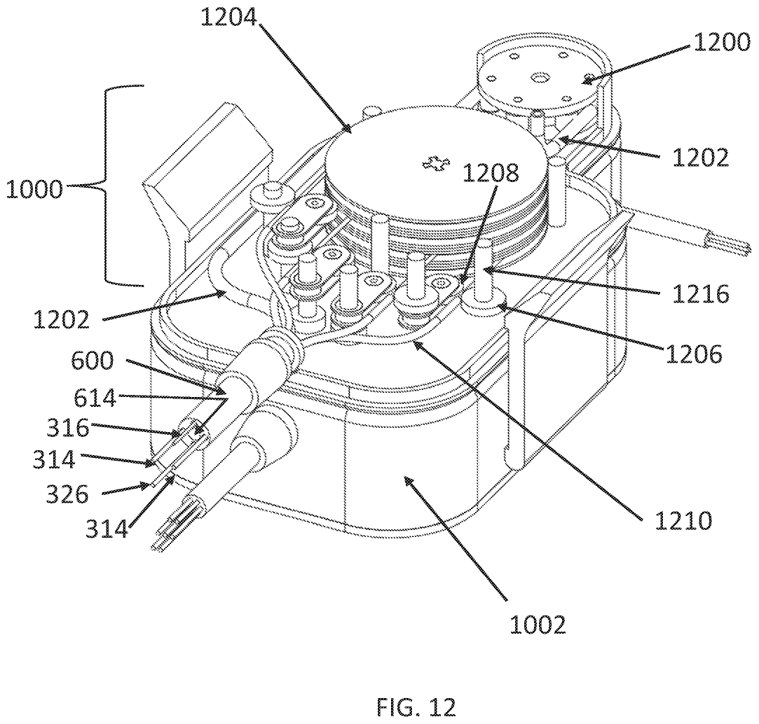

For further understanding, FIG. 12 depicts the interior of the disposable component (1000), with the bladder (1100) removed to depict the peristaltic pump (1200). The peristaltic pump (1200) is in operable communication with the bladder and lumen (614) via tubing (1202) or other suitable fluid transfer means.

Also as shown within the disposable component (1000) are a series of stacked and independently controllable wire reels (1204) for the guide wire (326), the two push wires (314), and the snare wire (316) which are all routed in passages surrounding the lumen (614) in the catheter (600). In one aspect, the wire reels (1204) include four wire reels that are dimensioned such that the wires (314, 316, and 326) wrap in a single plane within each reel to prevent entanglement. The four wire reels (1204) are stacked vertically and coaxially and are held in place prior to mounting to the reusable base component (1002) by means of peripheral posts that also can provide snubbing in operation if required.

The four wires (314, 316, and 326) are transported using any suitable mechanism or technique, a non-limiting example of which includes by means of elastomer-encased capstan rollers (1206) and pinch rollers (1208) in a manner similar to audio tape transport mechanisms or other similar devices. The pinch rollers (1208) can be spring-loaded or alternatively positioned at a fixed distance to the capstan roller (1206) to provide sufficient friction for deploying and retracting the wires (314, 316, and 326). The wires (314, 316, and 326) are routed between the capstan roller (1206) and the catheter (600) using any suitable mechanism or device, a non-limiting example of which includes rigid guide tubes (1210) whose openings are located close to the capstan rollers (1206), allowing the wires (314, 316, and 326) to be deployed into the catheter (600) as well as retracted therefrom.

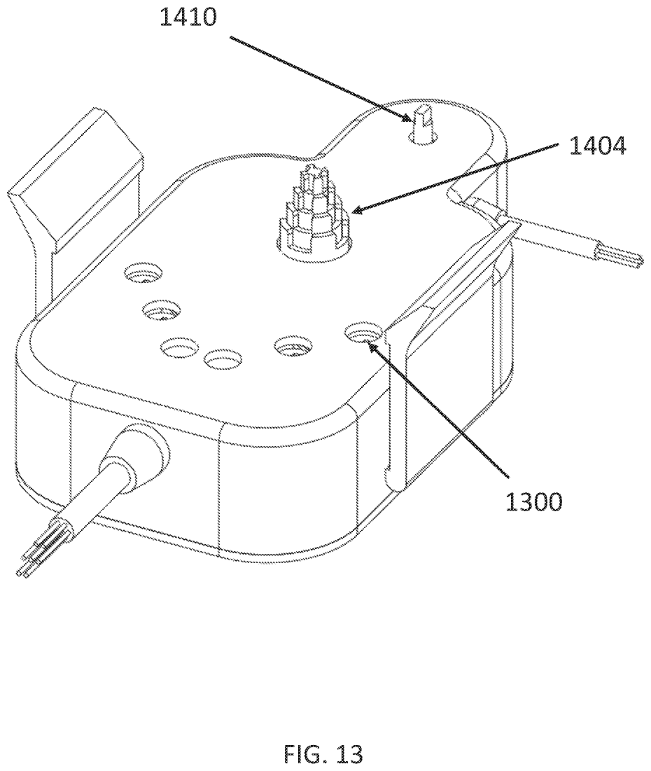

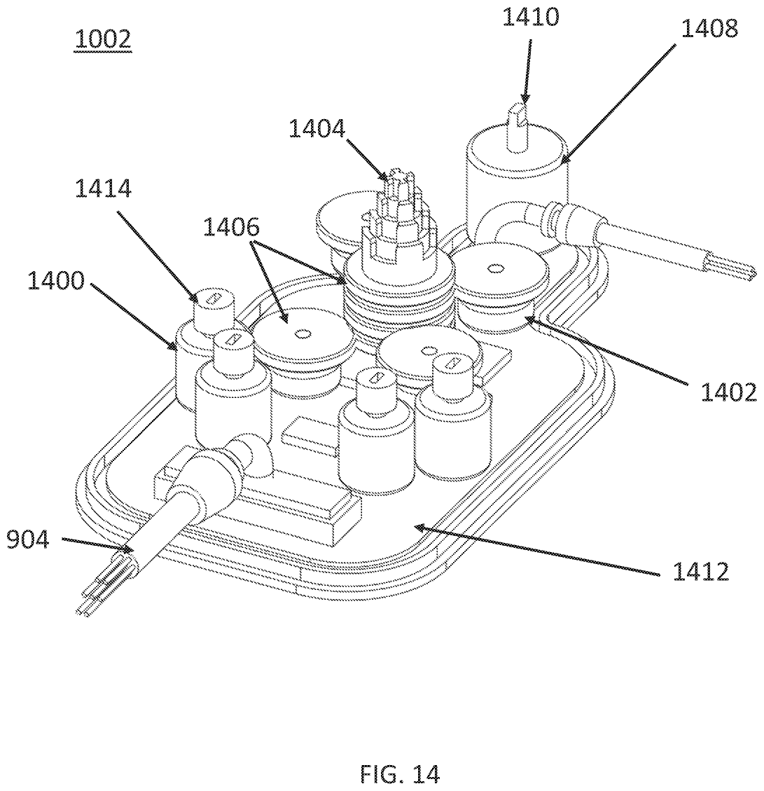

Referring to the interior-view illustration of the reusable base component (1002) as depicted in FIG. 14, the four capstan rollers (1206) are driven by four stepper motors (1400) and associated capstan drive shafts (1414) located for precise positioning and holding. In one aspect, the capstan roller (1206) central shaft (1216) passes through a hole (1300)(shown in FIG. 13) to selectively engage with the associated capstan drive shaft (1414). Similarly, the four wire reels (1204) are driven by four servo motors (1402) which maintain tension between the reels (1204) and the capstans (1206) and work in concert with the capstan stepper motors (1400) in deploying and retracting the wires (314, 316, and 326). Notably, the servo motors (1402) are operably connected with a series of axially aligned (i.e., coaxial) but independently operable wire wheel drive shafts (1404) via rollers (1406) or other suitable connection means (e.g., gears, etc.). The coaxial wheel drive shafts (1404) are used to independently control each of the reels (1204) and associated wires (314, 316, and 326). The coaxial wheel drive shafts (1404) for the wire reels (1204) are nested and engage the reels (1204) with mating features.

Further, the peristaltic pump (1200) is powered by a stepper motor (1408) housed within the base component (1002). Projecting from the stepper motor (1408) is a peristaltic motor drive shaft (1410) that is keyed to connect with and operate the peristaltic pump (1200). Also shown in the base component (1002) is a printed circuit board (PCBA) (1412) with circuits to control the motors as commanded by the hand controller with signals sent to the PCBA (1412) via an electrical cable (904) or other suitable signal transmission means (e.g., wireless, etc.).

As can be understood by those skilled in the art and referring to FIG. 13, the coaxial wheel drive shafts (1404) and peristaltic motor drive shaft (1410) are rotationally positioned electronically into an alignment to engage the disposable component (1000) prior to disposable component (1000) engagement. The wire reels (1204), capstans (1206) and the peristaltic pump (1200) are driven by keyed interfaces and their rotational positions in the disposable component (1000) are pre-positioned to engage the base component (1002) drive shafts (1404, 1414, and 1410, respectively).

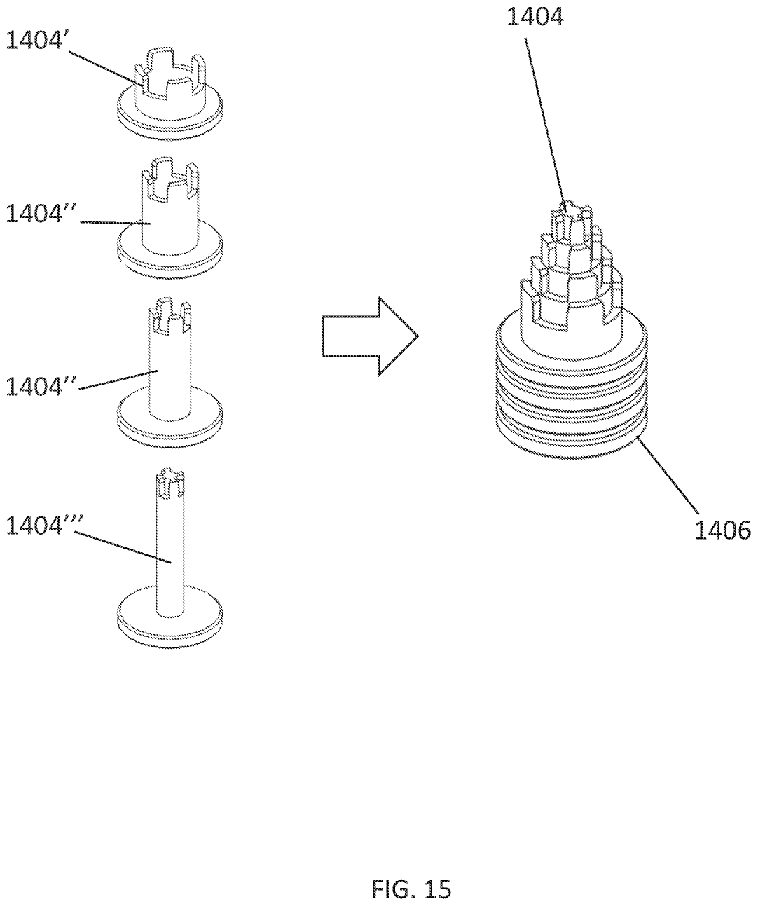

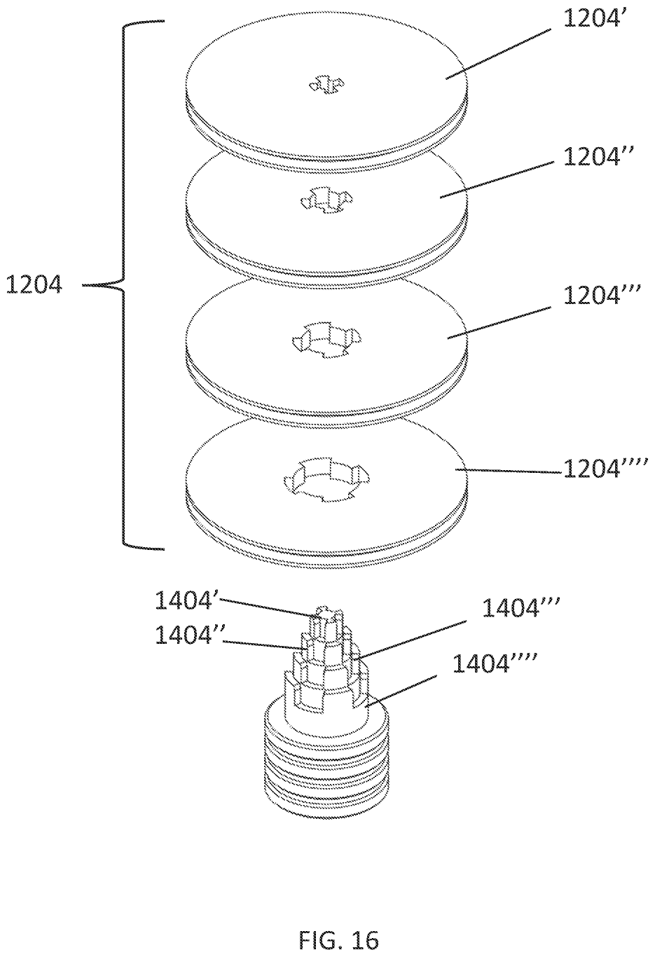

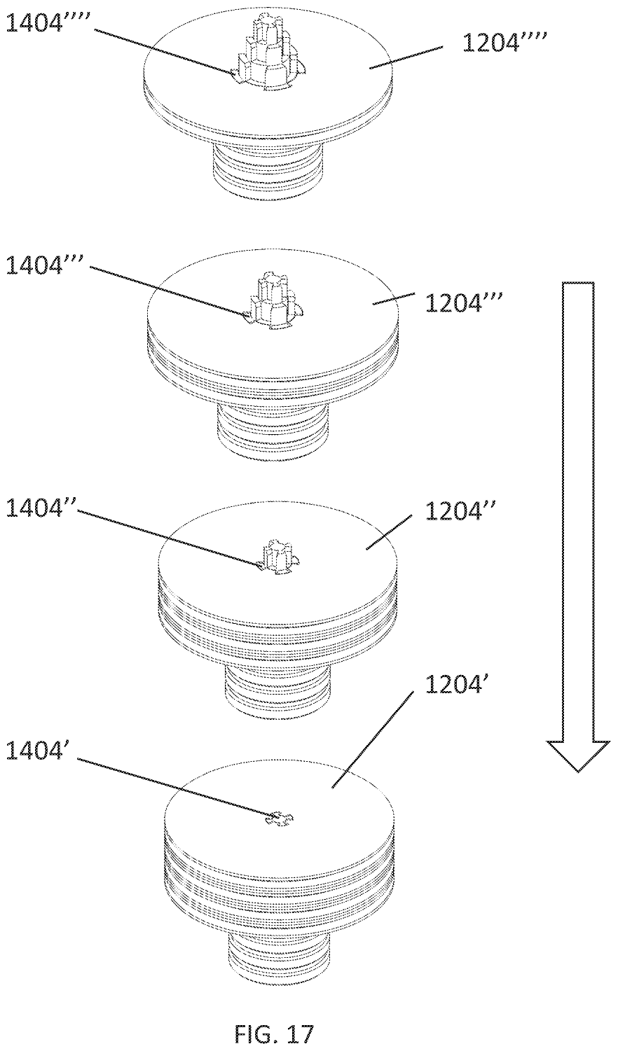

As noted above, the drive shafts (1404) are coaxial and independently controllable. For example and as depicted in FIG. 15, the drive shafts (1404) and rollers (1406) are formed of a series of nested drive shafts (1404′, 1404″, 1404′″, 1404′″). As shown in FIGS. 16 and 17, once the drive shafts (1404) are coaxially aligned, the independently controllable reels (1204′, 1204″, 1204′″, and 1204″″) can be progressively attached to the corresponding drive shaft (1404′, 1404″, 1404′″, 1404′). As shown, each reel (1204′, 1204″, 1204′″, and 1204″″) is keyed to an individual drive shaft (1404′, 1404″, 1404′, 1404′″) to allow for independent control of each associated reel (1204′, 1204″, 1204′, and 1204″″) and, thereby, each of the relevant wires.

It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed.

The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

The terms “patient” and “subject” are used interchangeably herein. These terms are intended to include all animal subjects, including mammals. Human patients/subjects are intended to be within the scope of the patients/subjects treated using the various embodiments of the inventive systems, apparatuses and methods described herein.

Finally, while this invention has been described in terms of several embodiments, one of ordinary skill in the art will readily recognize that the invention may have other applications in other environments. It should be noted that many embodiments and implementations are possible. Further, the following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”. Further, while particular method steps have been recited in a particular order, the method steps may occur in any desired order and fall within the scope of the present invention.

1. A system for implanting a gastric bypass device at a desired position within stomach of a patient, comprising:
a catheter assembly with a proximal end and a distal end, wherein the catheter assembly includes a hole at a center of the distal end;
a capsule body with a first end and a second end opposite the first end, wherein the capsule body is connected from the first end to the proximal end of the catheter assembly, the capsule body having a hollow lumen including a folded sleeve attached with the gastric bypass device;
a flexible catheter connected with the capsule body; and
a motorized controller attached with the flexible catheter and operably connected with the folded sleeve, wherein the motorized controller is operable for directing motion of the catheter assembly and causing expansion of the folded sleeve with a fluid, whereby expansion of the folded sleeve causes disengagement of the folded sleeve from the capsule body and the catheter assembly and allows the gastric bypass device to be released from the folded sleeve and implanted at the desired position in the stomach.

2. The system as set forth in claim 1, wherein the motorized controller includes a base station having a plurality of independently controllable and coaxially aligned drive drafts, each of the draft shafts having a reel affixed thereto.

3. The system as set forth in claim 2, wherein the controller further includes a bladder and peristaltic pump, the peristaltic pump being controllable for forcing fluid from the bladder into the folded sleeve.

4. The system as set forth in claim 3, wherein the flexible catheter further comprises:
a first hole at other side of the flexible catheter to receive a snare wire, wherein the snare wire is inserted in the first hole of the flexible catheter and received by the first end of the capsule body via the capsule cover; and
a pair of second holes at the other side of the flexible catheter to receive a push wire from each of the pair of second holes, wherein the push wires are inserted in each of the pair of second holes and received at the first end of the capsule body via the capsule cover, further wherein the snare wire engages with the push wires to retain the folded sleeve at the first end;
a third hole at the other side to receive a guide wire, wherein the guide wire is inserted in the third hole and received at the centre of the proximal end of the catheter assembly via the capsule body and the capsule cover; and
a central lumen at the other side for transferring the fluid to the folded sleeve within the capsule body via the capsule cover; and
wherein each of the snare wire, push wires, and guide wire are wrapped around a reel to allow for independent control thereof by the controller, such that the controller controls precise advancement and retraction of the guide wire, the push wires, the snare wire and transfer of the fluid into the folded sleeve, thereby expanding and disengaging the folded sleeve from the capsule body and the catheter assembly and allows the gastric bypass device to be released from the folded sleeve and implant the gastric bypass device at the desired position in the stomach.

5. The system as set forth in claim 4, wherein the catheter assembly further comprises:
a nose cone insert with a front portion and a back portion, wherein the back portion is connected with the capsule body and retains the folded sleeve in position by the snare wire and the push wire, further wherein the front portion is fixed to the back portion from a first side and allows the guide wire to pass through the hole in the second side opposite the first side; and
a nose cone enclosing the nose cone insert, wherein the nose cone includes an opening to overlap with the hole of the nose cone insert.

6. The system as set forth in claim 5, wherein each of the nose cone insert and the nose cone are made of polycaprolactone material.

7. The system as set forth in claim 6, wherein the back portion of the nose cone insert disengages from the folded sleeve.

8. The system as set forth in claim 1, further comprising a spring wire support within an internal periphery of the capsule body to hold the gastric bypass device.

9. The system as set forth in claim 8, wherein expansion of the folded sleeve allows the spring wire support to disengage from the folded sleeve and release the gastric bypass device.

10. The system as set forth in claim 8, wherein the spring wire support is serpentine in shape and made up of nitinol material.

11. The system as set forth in claim 1, wherein the gastric bypass device takes the shape of a funnel.

12. The system as set forth in claim 1, wherein position of the nose assembly is continuously monitored through fluoroscope.

13. The system as set forth in claim 1, wherein the fluid is either water or saline solution.

14. The system as set forth in claim 1, wherein the fluid is either air or carbon dioxide gas.

15. The system as set forth in claim 1, wherein the controller further includes a bladder and peristaltic pump, the peristaltic pump being controllable for forcing fluid from the bladder into the folded sleeve.

16. A method for implanting a gastric bypass device at a desired position within a stomach of a patient, the method comprising acts of:
advancing a guide wire with a motorized controller through an esophagus into the stomach and upper digestive tract of the patient from a catheter assembly connected with a controller;
deploying a capsule body connected with the catheter assembly at one end near the desired position;
advancing a pair of push wires and a snare wire in the capsule body with the controller;
withdrawing the capsule body from the catheter assembly to unfold a sleeve with a gastric bypass device within the capsule body;
injecting a fluid from a fluid transfer tube of the controller via a lumen of a flexible catheter attached to a capsule cover;
inflating the sleeve within the capsule body with the fluid to form an inflated sleeve;
disengaging the inflated sleeve from the capsule body;
removing the guide wire followed by the snare wire and the pair of push wires by the controller to release the catheter assembly from the inflated sleeve;
releasing a spring wire support holding the gastric bypass device from the inflated sleeve;
emerging the gastric bypass device to acquire a funnel shape; and
implanting the gastric bypass device within the stomach of the patient at the desired position.
